(19) United States
(12) Patent Application Publication
(10) Pub. No.: US 2022/0241429 A1
(43) Pub. Date: Aug. 4, 2022

(54) Antifolate-Carrying Nanoparticles and Their Use in Medicine

(71) Applicant: Midatech Ltd, Cardiff, South Glamorgan (GB)

(72) Inventors: Martina McAteer, Cardiff (GB); Tom Coulter, Cardiff (GB); Yao Ding, Cardiff (GB); John Porter, Cardiff (GB)

(21) Appl. No.: 17/413,472

(22) PCT Filed: Dec. 13, 2019
(86) PCT No.: PCT/EP2019/085203

§ 371 Date: Jun. 11, 2021

(30) Foreign Application Priority Data
Dec. 14, 2018 (GB) 1820470.1

(51) Int. Cl. A61K 47/69; A61K 47/54

(52) U.S. Cl. CPC A61K 47/6929; A61K 47/545; A61K 47/549

(57) ABSTRACT

The present invention provides a nanoparticle comprising: a core comprising a metal and/or a semiconductor; and a plurality of ligands covalently linked to the core, wherein said ligands comprise: (i) at least one dilution ligand comprising a carbohydrate, glutathione or an ethylene glycol-containing moiety; and (ii) a ligand of the formula D-L₁-Z-L₂, wherein D comprises an antifolate drug or folic acid, L₁ comprises a first linker portion comprising a C2-C12 glycol and/or C2-C12 alkyl chain, L₂ comprises a second linker portion comprising a C2-C12 glycol and/or C2-C12 alkyl chain, wherein L₁ and L₂ may be the same or different, and wherein Z represents a carbonyl-containing group linking L₁ and L₂, and wherein L₂ is coupled to said core. Also provided are pharmaceutical compositions comprising such nanoparticles, medical uses thereof and methods for producing the nanoparticles.

This application claims priority from GB1820470.1 filed 14 Dec. 2018, the contents and elements of which are herein incorporated by reference for all purposes.

### FIELD OF THE INVENTION

The present invention relates to nanoparticles as vehicles for delivery of active agents to specific tissue types or locations, particularly for use in medicine, and includes methods for treatment of inflammatory, autoimmune and proliferative disorders, including cancers. Pharmaceutical compositions, including topical gel formulations, and methods for their use are also disclosed.

### BACKGROUND TO THE INVENTION

The present invention is directed at compositions and products, and methods of making and administering such compositions and products, including for the treatment of mammals and particularly humans.

Antifolates are a class of antimetabolite medications that antagonise the actions of folic acid (vitamin B9), for example by the inhibition of dihydrofolate reductase (DHFR). Antifolates inhibit cell division, DNA/RNA synthesis and repair and protein synthesis. A variety of antifolates have found use in the treatment of cancers (e.g. methotrexate for haematological malignancies and osteosarcoma, pemetrexed for non-small cell lung carcinoma and mesothelioma) and inflammatory disorders (e.g. methotrexate for psoriasis and rheumatoid arthritis). Certain antifolates preferentially target microbial DHFR and find use as antimicrobials or antimalarials (e.g. trimethoprim or pyrimethamine).

Cellular uptake of folic acid is mediated by folate receptors such as folate receptor alpha and folate receptor beta. Folate receptor alpha is overexpressed on a number of epithelial-derived tumours including ovarian, breast, renal, lung, colorectal and brain tumours. Agents such as antibodies that bind the folate receptor are being developed for targeted therapy and diagnosis of cancers, e.g. farletuzumab for ovarian cancer.

Nanoparticle-based delivery of therapeutic agents may be employed to improve targeting, penetration (e.g. skin penetration) or other properties of the agent being delivered. Nanoparticles also find use in imaging and diagnosis. For example, Zwicke et al., 2012, Nano Rev, Vol. 3, 10.3402, reviews the use of the folate receptor for active targeting of cancer nanotherapeutics. Hu et al., 2014, Theranostics, 4(2), pp. 142-153, describes a folate-receptor-targeting gold nanocluster that acts as a fluorescence enzyme mimetic for tumour molecular colocalisation diagnosis. The gold nanoclusters were formed by reacting bovine serum albumin (BSA)-capped gold nanoclusters with folic acid using EDC/NHS coupling in anhydrous dimethyl sulfoxide (see FIG. 1 of Hu et al.).

Conjugation of antifolates, such as methotrexate and pemetrexed, to gold nanoparticles has also been reported. US2015/0231077 describes gold nanoparticles passivated with amine-containing molecules, including MTX. Stolarczyk et al., 2017, Eur J Pharm Sci, Vol. 109, pp. 13-20, describes pemetrexed conjugated gold nanoparticles in which pemetrexed was proposed to interact with the gold surface by the carboxylic acid group. Chen et al., Molecular Pharmaceutics, 2007, Vol. 4, No. 5, pp. 713-722, describes MTX adsorbed to 13 nm colloidal gold nanoparticles (see scheme 1) and subsequent assessment of the cytotoxic effect of MTX-AuNP on various cancer cells. Tran et al., Biochemical Engineering Journal, 2013, Vol. 78, pp. 175-180, describes fabrication of methotrexate-conjugated gold nanoparticles via a one-pot synthesis, and subsequent in vitro testing of MTX-AuNPs against cancer cells. Bessar et al., Colloids and Surfaces B: Biointerfaces, 2016, Vol. 141, pp. 141-147, describes non-covalent loading of MTX onto water-soluble gold nanoparticles functionalised with sodium 3-mercapto-1-propansulfonate (Au-3MPS) and proposes that Au-3MPS@MTX could be suitable as a topical therapy in psoriasis patients. The loading efficiency of MTX on Au-3MPS was assessed in the range of 70-80%, with a fast release (80% in one hour). Fratoddi et al., Nanomedicine: Nanotechnology, Biology and Medicine, 2019, Vol. 17, pp. 276-286 describe effects of topical Au-3MPS@MTX in cutaneous inflammatory mouse model.

Despite these advances, there remains an unmet need for further nanoparticle delivery systems for targeting the folate receptor and/or delivering antifolates, e.g. for treatment of cancers, inflammatory and autoimmune disorders. In particular, nanoparticles that exhibit improved loading (e.g. greater density of antifolate or folic acid payload and/or greater loading efficiency) and pharmaceutical compositions thereof, remain an unmet need. The present invention seeks to provide solutions to these needs and provides further related advantages.

### BRIEF DESCRIPTION OF THE INVENTION

Broadly, the present invention relates to antifolate-carrying nanoparticles and compositions thereof that find use in medicine. The present inventors have surprisingly found that a modification of a structural group (carboxylic acid group) common to folic acid and the class of antifolates, whereby a linker is attached to said group prior to conjugation of the compound to a nanoparticle, significantly enhanced the loading of the compound on the nanoparticle. As described further herein, a modified form of methotrexate, having an ethylene glycol chain with a terminal amine exhibited average loading of up to 10 methotrexate containing ligands or more per core, whereas coupling unmodified methotrexate to an amine terminal linker on the nanoparticle via a carboxylic acid group on the methotrexate molecule exhibited average loading of 5 or fewer methotrexate containing ligands per core. In addition to improving loading, the modification of methotrexate resulted in a more homogeneous population of nanoparticles, presumably as a result of a more controlled site of attachment of the methotrexate to the nanoparticle. Moreover, the linker modification (e.g. -(EG)₃-NH₂ modification) to an antifolate, such as methotrexate, may be used to modulate the solubility of the construct and/or to alter local ordering of the corona ligands. It is also thought that the conjugated nanoparticles herein demonstrate synergistic activity relative to the activity of the ligand alone and the nanoparticle alone.

Accordingly, in a first aspect the present invention provides a nanoparticle comprising:
a core comprising a metal and/or a semiconductor; and
a plurality of ligands covalently linked to the core, wherein said ligands comprise:
(i) at least one dilution ligand comprising a carbohydrate, glutathione or an ethyleneglycol-containing moiety; and
(ii) a ligand of the formula D-L₁-Z-L₂, wherein D comprises an antifolate drug or folic acid, L₁ comprises a first linker portion comprising a C2-C12 glycol and/or C1-C12 or C2-C12 alkyl chain, L₂ comprises a second linker portion comprising a C2-C12 glycol and/or C1-C12 or C2-C12 alkyl chain, wherein L₁ and L₂ may be the same or different, and wherein Z represents a divalent linker group of up to 10 atoms linking L₁ and L₂, and wherein Z comprises at least 2 heteroatoms and L₂ is coupled to said core.

In some embodiments D comprises the following structure:

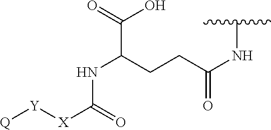

wherein;
X is a 3 to 8 membered (e.g. 5 or 6 membered) carbocyclic, heterocyclic, carboaromatic or heteroaromatic ring,
Y is a linker group having 1 to 20 atoms comprising one or more atoms selected from H, C, N, O and S;
wherein Y is optionally substituted by one or more groups having 1 to 20 atoms comprising of one or more atoms selected from H, C, N, O and S, and
Q is a fused bicyclic heterocyclic or heteroaromatic ring optionally substituted with one or more groups selected from amino, hydroxyl, carbonyl, methyl, ethyl, propyl, isopropyl, butyl and isobutyl. In particular, Q may be a substituted pteridine.

In some embodiments D comprises an antifolate drug selected from the group consisting of: methotrexate, pemetrexed, ralitrexed and pralatrexate.

In some embodiments D is selected from the following structures;

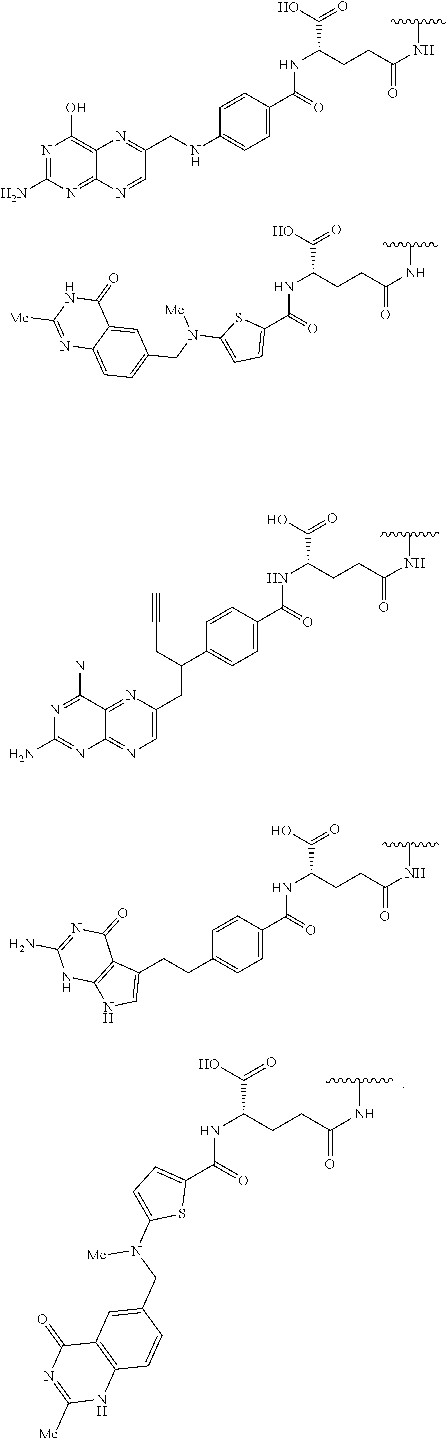

In some embodiments Z comprises a 3-10 membered carboaromatic, a 3-10 membered carbocycle, a 3-10 membered heterocycle, a 3-10 membered heteroaromatic, an imide, an amidine, a guanidine, a 1,2,3-triazole, a sulfoxide, a sulfone, a thioester, a thioamide, a thiourea, an amide, an ester, a carbamate, a carbonate ester or a urea. In some embodiments Z represents a carbonyl-containing group. In some embodiments Z comprises between 1 to 6 atoms. Preferably, Z comprises an amide.

In some embodiments L₁ comprises —(OCH₂CH₂)ₚ— and L₂ comprises —(OCH₂CH₂)_{q}— and wherein each of p and q is a number in the range 2 to 10 (e.g. 2, 3, 4, 5, 6, 7, 8, 9 or 10), and wherein p and q may be the same or different. In particular p may be 3 and/or q may be 8.

In some embodiments D-L₁-Z-L₂ is of the formula:

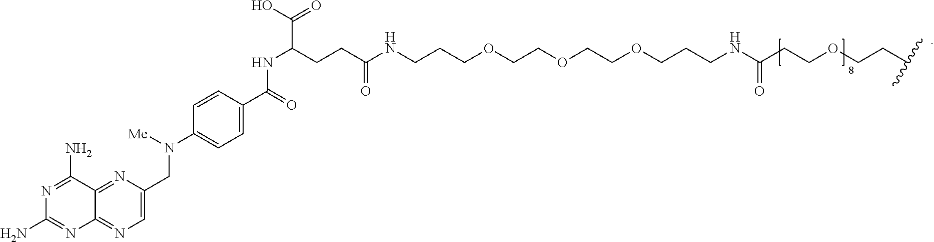

In some embodiments D-L₁-Z-L₂ is of the formula:

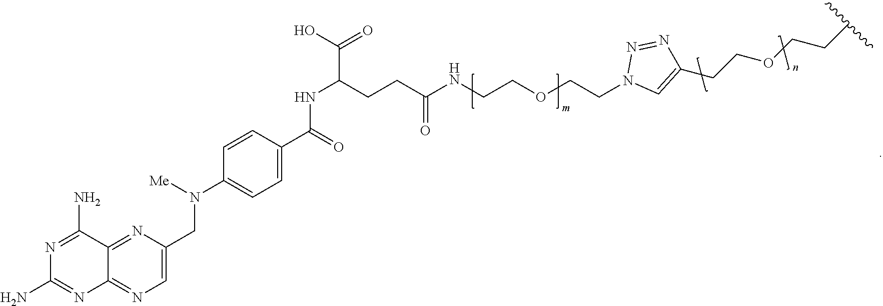

In some embodiments D-L₁-Z-L₂ is of the formula:

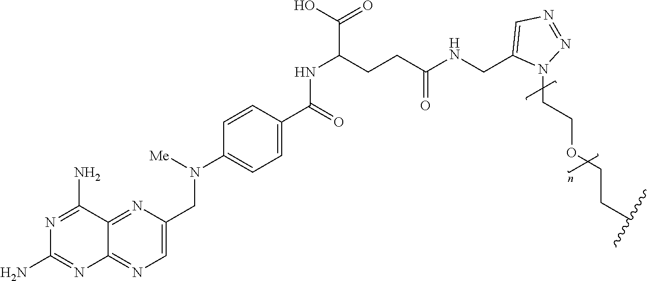

In some embodiments D-L₁-Z-L₂ is of the formula:

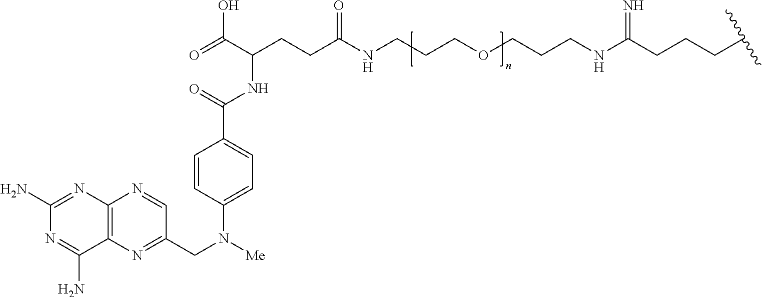

In some embodiments L₂ is bound to the core via a terminal sulphur atom.

In particular, D-L₁-Z-L₂ may be of the formula:

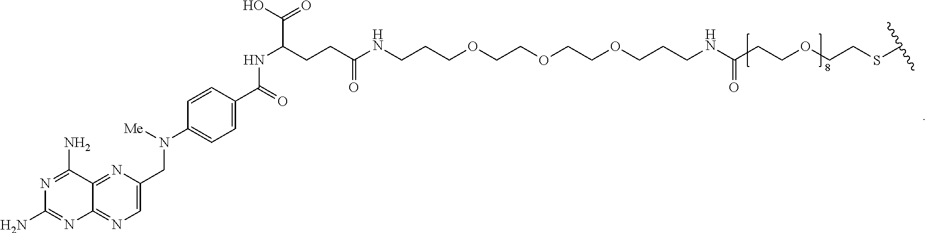

In some embodiments, D-L₁-Z-L₂ may be of the formula:

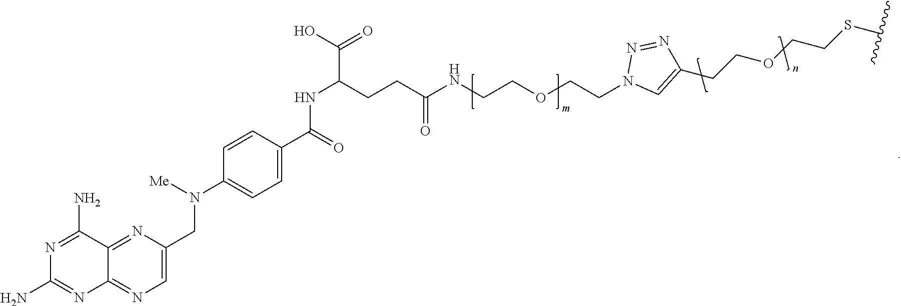

wherein n and m are independently 1, 2, 3, 4, 5, 6, 7, 8, 9 or 10.

In some embodiments, D-L₁-Z-L₂ may be of the formula:

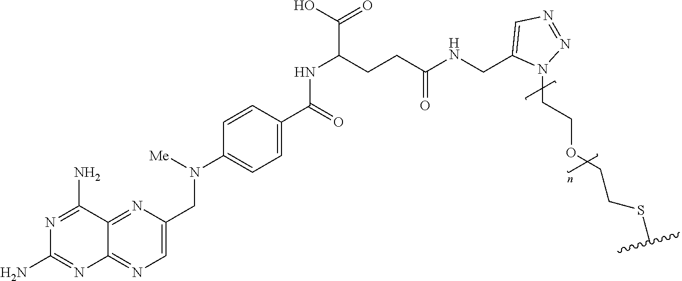

wherein n is an integer of between 1 and 15.

In some embodiments, D-L₁-Z-L₂ may be of the formula:

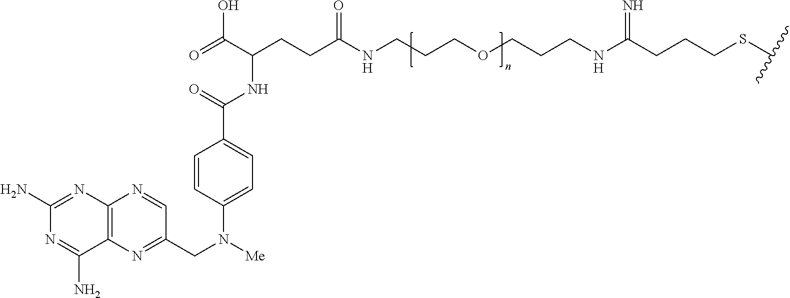

wherein n is an integer of between 1 and 15.

In some embodiments, the nanoparticle may be of the formula: [dilution ligand]ₛ[D-L₁-Z-L₂-S]ₜ@Au, wherein s and t are independently numbers >1. In some cases s may be >20. In some cases t may be >3, e.g., >5 or even >10.

Typically, the nanoparticle will have unreacted linker ligands that have not had D coupled to them. Accordingly, in some embodiments the nanoparticle may be of the formula: [dilution ligand]ₛ[D-L₁-Z-L₂-S]ₜ[COOH-L₂-S]ᵤ@Au, wherein s, t and u are independently numbers >1. In some cases s may be >20, e.g., >30. In some cases t may be >3, e.g., >5 or even >10. In some cases u may be >10, e.g., >20.

In some embodiments the dilution ligand comprises a carbohydrate which is a monosaccharide or a disaccharide. For example, the dilution ligand may comprise galactose, glucose, mannose, fucose, maltose, lactose, galactosamine and/or N-acetylglucosamine.

In particular, the dilution ligand may comprise 2′-thioethyl-α-D-galactopyranoside or 2′-thioethyl-R-D-glucopyranoside.

In some embodiments the core comprises a metal selected from the group consisting of: Au, Ag, Cu, Pt, Pd, Fe, Co, Gd, Zn or any combination thereof. In particular, the core may comprise gold.

In some embodiments the nanoparticle may be of the formula: [α-galactose-C2-S]ₛ[MTX-L₁-Z-L₂-S]ₜ@Au, wherein s and t are independently numbers >1. In some cases s may be >20. In some cases t may be >3, e.g., >5 or even >10.

In some embodiments the nanoparticle may be of the formula: [α-galactose-C2-S]ₛ[MTX-L₁-Z-L₂-S]ₜ[COOH-L₂-S]ᵤ@Au, wherein s, t and u are independently numbers >1. In some cases s may be >20, e.g., >30. In some cases t may be >3, e.g., >5 or even >10. In some cases u may be >10, e.g., >20.

In some embodiments the diameter of the core is in the range 1 nm to 5 nm, e.g., 2 to 4 nm.

In some embodiments the diameter of the nanoparticle including its ligands is in the range 3 nm to 50 nm.

Diameter may be taken to be the longest diameter of the nanoparticle core or nanoparticle. Determination may be made using, for example, electron microscopy or dynamic light scattering (DLS).

In some embodiments the total number of ligands per core is in the range 20 to 200.

In some embodiments the number of ligands of said formula D-L₁-Z-L₂ per core is at least 10, optionally at least 15.

In some embodiments the nanoparticle has the following structure:

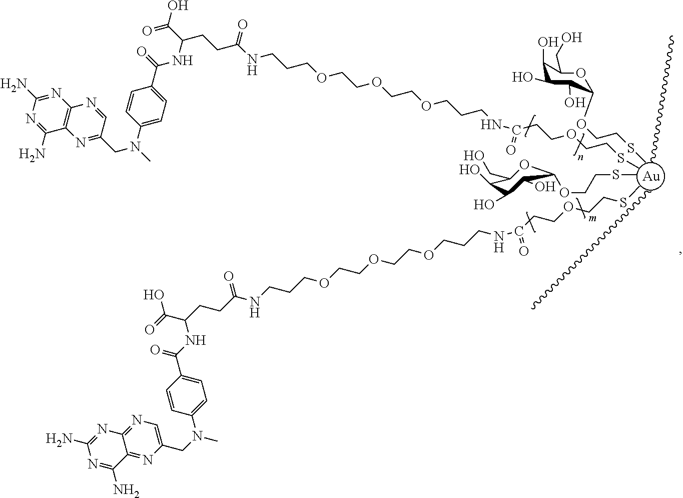

wherein the total number of ligands per core is at least 5, and the total number of methotrexate-containing ligands per core is at least 3. Preferably, the total number of ligands per core is at least 10, 15 or 20. Preferably, the total number of methotrexate-containing ligands per core is at least 5, 10 or 15.

In some embodiments the nanoparticle has the following structure:

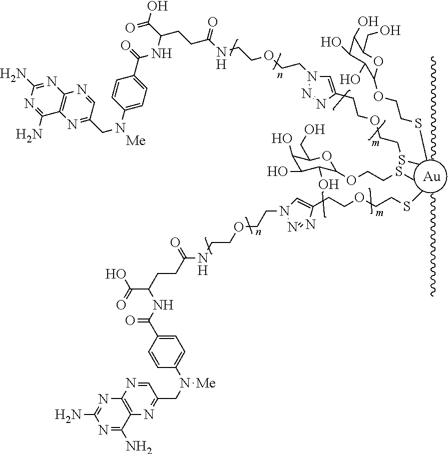

wherein n and m are independently 1, 2, 3, 4, 5, 6, 7, 8, 9 or 10, the total number of ligands per core is at least 5, and the total number of methotrexate-containing ligands per core is at least 3. Preferably, the total number of ligands per core is at least 10, 15 or 20. Preferably, the total number of methotrexate-containing ligands per core is at least 5, 10 or 15.

In some embodiments the nanoparticle has the following structure:

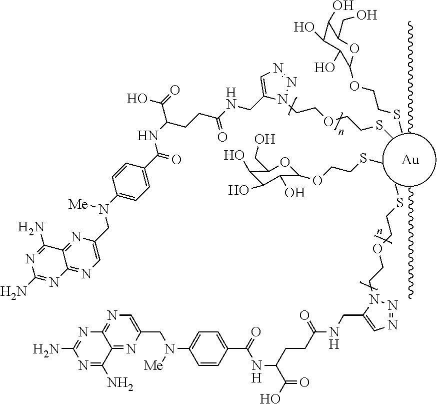

wherein n is an integer of between 1 and 15, the total number of ligands per core is at least 5, and the total number of methotrexate-containing ligands per core is at least 3. Preferably, the total number of ligands per core is at least 10, 15 or 20. Preferably, the total number of methotrexate-containing ligands per core is at least 5, 10 or 15.

In some embodiments the nanoparticle has the following structure:

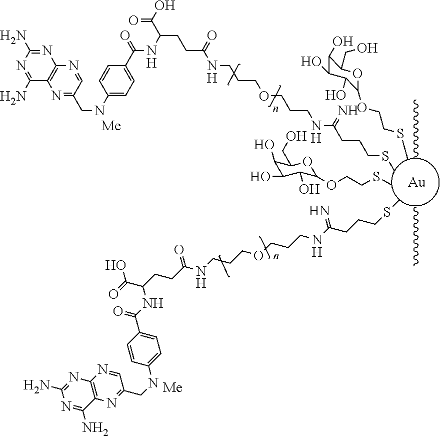

wherein n is an integer of between 1 and 15, the total number of ligands per core is at least 5, and the total number of methotrexate-containing ligands per core is at least 3. Preferably, the total number of ligands per core is at least 10, 15 or 20. Preferably, the total number of methotrexate-containing ligands per core is at least 5, 10 or 15.

In some embodiments the plurality of ligands further comprises a therapeutically active agent and/or a detectable moiety. This may be particularly useful wherein D comprises a folate receptor binding agent (e.g. folic acid or an antifolate that binds to a folate receptor, such as methotrexate) because D may cause the nanoparticle to be targeted to folate receptor expressing cells or tissue and may facilitate uptake of the nanoparticle by such cells or tissue (e.g. tumour cells or tumour tissue). The therapeutically active agent may comprise an anti-cancer agent. In this way the nanoparticle may be employed for folate receptor targeted therapy, e.g., of a folate receptor overexpressing tumour. Likewise, where the nanoparticle has a ligand that is a detectable moiety (e.g. a fluorescent label), the nanoparticle may find use in detection of folate receptor overexpressing cells or tissue, such as in imaging, diagnosis and/or treatment monitoring of a cancer that expresses folate receptor.

In some embodiments the anti-cancer agent may be selected from the group consisting of: a maytansinoid (e.g. maytansinoid DM1 or maytansinoid DM4), doxorubicin, irinotecan, Platinum (II), Platinum (IV), temozolomide, carmustine, camptothecin, docetaxel, sorafenib, monomethyl auristatin E (MMAE) and panobinostat.

In a second aspect the present invention provides a pharmaceutical composition comprising a plurality of nanoparticles of the first aspect of the invention and at least one pharmaceutically acceptable carrier or diluent.

In some embodiments the pharmaceutical composition may be in the form of a gel, optionally a hydrogel.

In some embodiments said gel is selected from the group consisting of: Carbopol® 980, Carbopol® 974 and Carbopol® ETD 2020.

In some embodiments the concentration of said antifolate drug in said gel is in the range 0.5 mg/mL to 10 mg/mL, optionally about 2 mg/mL.

In some embodiments the nanoparticle core is of gold and the concentration of gold in said gel is in the range 1 mg/mL to 20 mg/mL, optionally about 4 mg/mL.

In some embodiments the composition is for topical administration.

In some embodiments the composition is for systemic administration (e.g. intravenous or subcutaneous injection).

In a third aspect the present invention provides a nanoparticle of the first aspect of the invention or a pharmaceutical composition of the second aspect of the invention for use in medicine.

In a fourth aspect the present invention provides a nanoparticle of the first aspect of the invention or a pharmaceutical composition of the second aspect of the invention for use in the treatment of a proliferative disorder, an inflammatory disorder or an autoimmune disease in a mammalian subject. The proliferative disorder may be a cancer, such as a folate receptor expressing cancer. In particular the cancer may be a cancer of cancers of the ovary, breast, pleura, lung, cervix, endometrium, kidney, bladder or brain.

In a fifth aspect the present invention provides a method of treating a proliferative disorder, an inflammatory disorder or an autoimmune disease in a mammalian subject, comprising administering a nanoparticle of the first aspect of the invention or a pharmaceutical composition of the second aspect of the invention to the subject in need of therapy. The proliferative disorder may be a cancer, such as a folate receptor expressing cancer. In particular the cancer may be a cancer of cancers of the ovary, breast, pleura, lung, cervix, endometrium, kidney, bladder or brain.

In a sixth aspect the present invention provides use of a nanoparticle of the first aspect of the invention or a pharmaceutical composition of the second aspect of the invention in the preparation of a medicament for use in a method of the fifth aspect of the invention.

In a seventh aspect the present invention provides an article of manufacture comprising:
a nanoparticle of the first aspect of the invention or a pharmaceutical composition of the second aspect of the invention;
a container for housing the nanoparticle or pharmaceutical composition; and
an insert or label.

In some embodiments the insert and/or label provides instructions, dosage and/or administration information relating to the use of the nanoparticle or pharmaceutical composition in the treatment of a proliferative disorder, an inflammatory disorder or an autoimmune disease in a mammalian subject. The proliferative disorder may be a cancer, such as a folate receptor expressing cancer. In particular the cancer may be a cancer of the ovary, breast, pleura, lung, cervix, endometrium, kidney, bladder or brain.

In an eighth aspect the present invention provides a compound of the formula D-L₁-R₁, wherein D comprises an antifolate drug or folic acid, L₁ comprises —(OCH₂CH₂)ₚ—, wherein p is an integer in the range 1 to 10 (e.g. 1, 2, 3, 4, 5, 6, 7, 8, 9 or 10), and wherein R₁ comprises an amine group.

In some embodiments the compound has the structural formula:

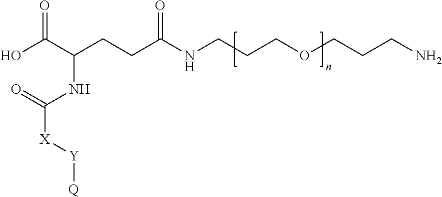

wherein;
   X is a 3 to 8 membered (e.g. 5 or 6 membered) carbocyclic, heterocyclic, carboaromatic or heteroaromatic ring,
   Y is a linker group having 1 to 20 atoms comprising one or more atoms selected from H, C, N, O and S;
   wherein Y is optionally substituted by one or more groups having 1 to 20 atoms comprising of one or more atoms selected from H, C, N, O and S, and
   Q is a fused bicyclic heterocyclic or heteroaromatic ring optionally substituted with one or more groups selected from amino, hydroxyl, carbonyl, methyl, ethyl, propyl, isopropyl, butyl and isobutyl.

In some embodiments the compound has the structural formula:

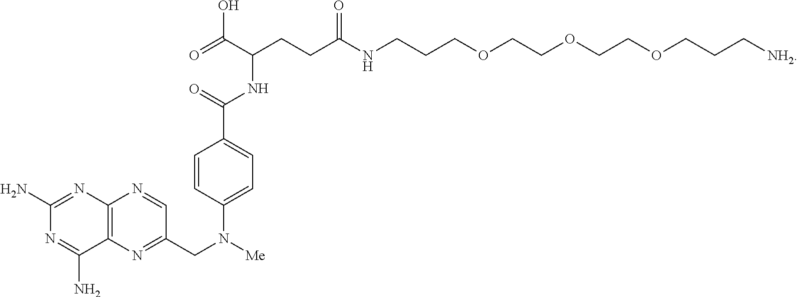 [SMILES: CN(Cc1cnc2nc(N)nc(N)c2n1)c1ccc(C(=O)NC(CCC(=O)NCCCOCCOCCOCCCN)C(=O)O)cc1]

In a ninth aspect, the invention provides a process for the production of a compound of formula (h) comprising the following steps;

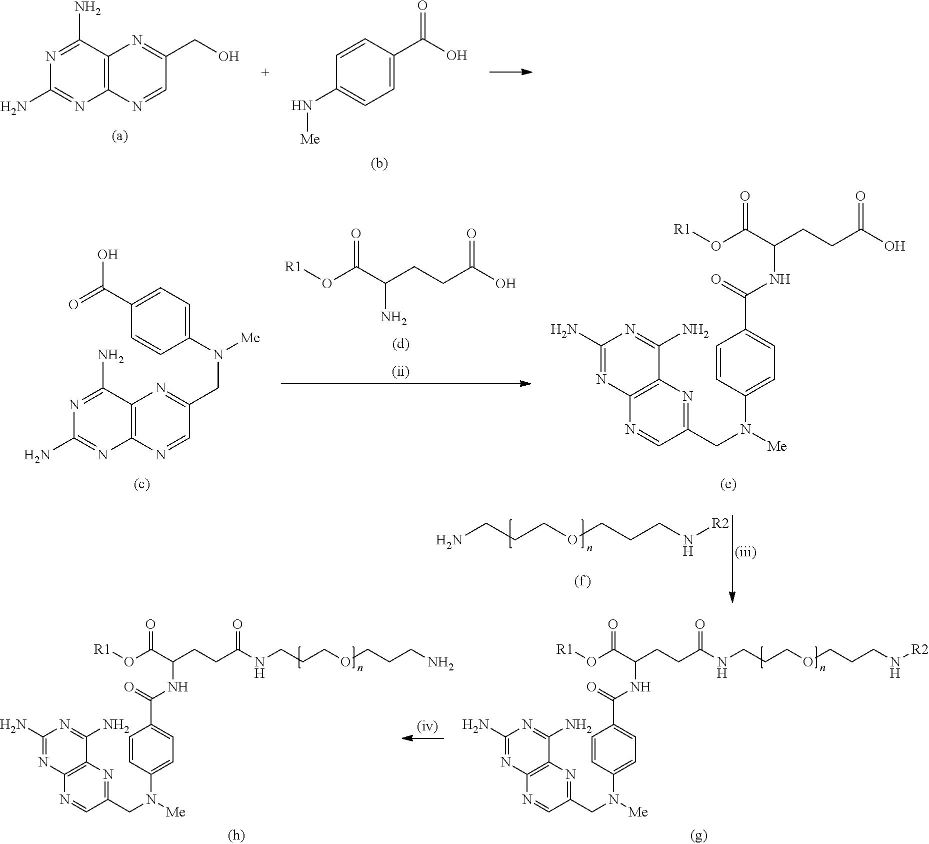

(i) halogenation of an alcohol of formula (a) to afford an halogen compound (al) that is used in a displacement reaction with an amine of formula (b) to afford an compound of formula (c);
(ii) performing an amide coupling with compounds of formulae (c) and (d) to afford an amide of formula (e),
(iii) performing an amide coupling with compounds of formulae (e) and (f) to afford an amide of formula (g),
(iv) removing the amine and carboxylic acid protecting groups of the compound of formula (g) to afford a compound of formula (h),
wherein R1 is a carboxylic acid protecting group and R2 is an amine protecting group.

In some embodiments, R1 and R2 are each acid labile protecting groups. Preferably, R1 and R2 are tert-butyloxycarbonyl protecting groups. In some embodiments n is 3. In some embodiments the halogenation is chlorination, bromination or iodation. Preferably, the halogenation is bromination by triphenylphosphine dibromide.

In a tenth aspect the present invention provides a process for the production of a compound of formula (h) comprising the following steps;

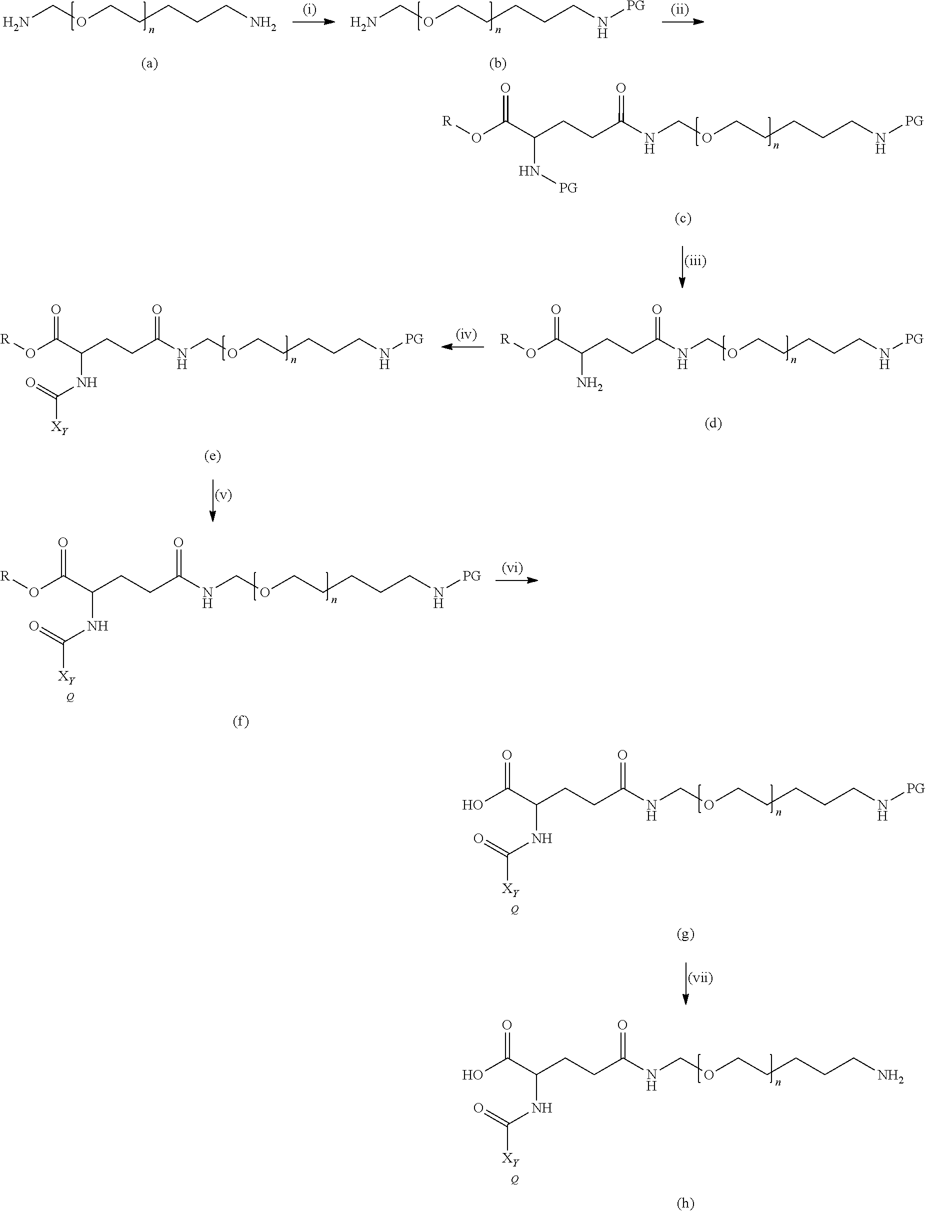

(i) protecting an amine of formula (a) with a first protecting group to afford a compound of formula (b);
(ii) performing an amide coupling with a compound of formula (b) to afford an amide of formula (c),
(iii) removing a second amine protecting group from the amide of formula (c) to afford a compound of formula (d),
(iv) performing an amide coupling with the compound of formula (d) to afford an amide of formula (e),
(vi) coupling a reagent comprising Q with the compound of formula (f) to afford a compound of formula (f),
(vii) hydrolysing the ester of the compound of formula (f) to afford a compound of formula (g), and
(viii) removing the first protecting group from the compound of formula (g) to afford the compound of formula (h), wherein,
R is a hydrocarbon group comprising 1 to 6 carbon atoms,
X is a 3 to 8 membered carbocyclic, heterocyclic, carboaromatic or heteroaromatic ring,
Y is a linker group having 1 to 20 atoms comprising one or more atoms selected from H, C, N, O and S;
wherein Y is optionally substituted by one or more groups having 1 to 20 atoms comprising of one or more atoms selected from H, C, N, O and S, and
Q is a fused bicyclic heterocyclic or heteroaromatic ring optionally substituted with one or more groups selected from amino, hydroxyl, carbonyl, methyl, ethyl, propyl, isopropyl, butyl and isobutyl.

In some embodiments the process is for the production of 4-[(3-{2-[2-(3-aminopropoxy)ethoxy]ethoxy}propyl)carbamoyl]-2-[(4-{[(2,4diaminopteridin6yl)methyl](methyl)amino}phenyl)formamido]butanoic acid, comprising:

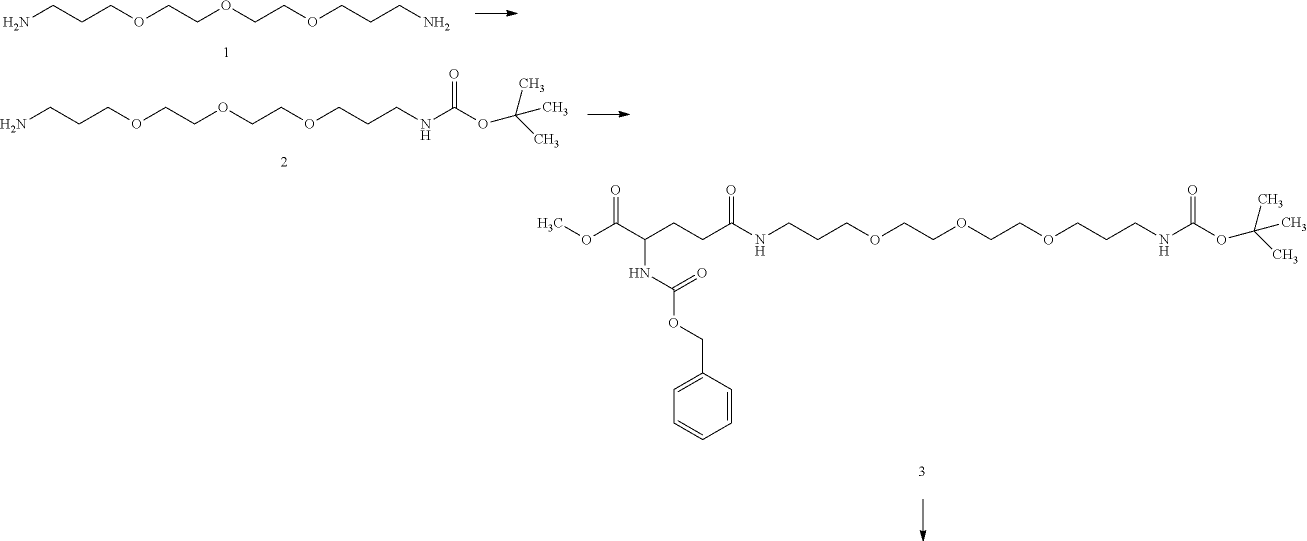 [SMILES: NCCCOCCOCCOCCCN ; CC(C)(C)OC(=O)NCCCOCCOCCOCCCN ; COC(=O)C(CCC(=O)NCCCOCCOCCOCCCNC(=O)OC(C)(C)C)NC(=O)OCc1ccccc1]

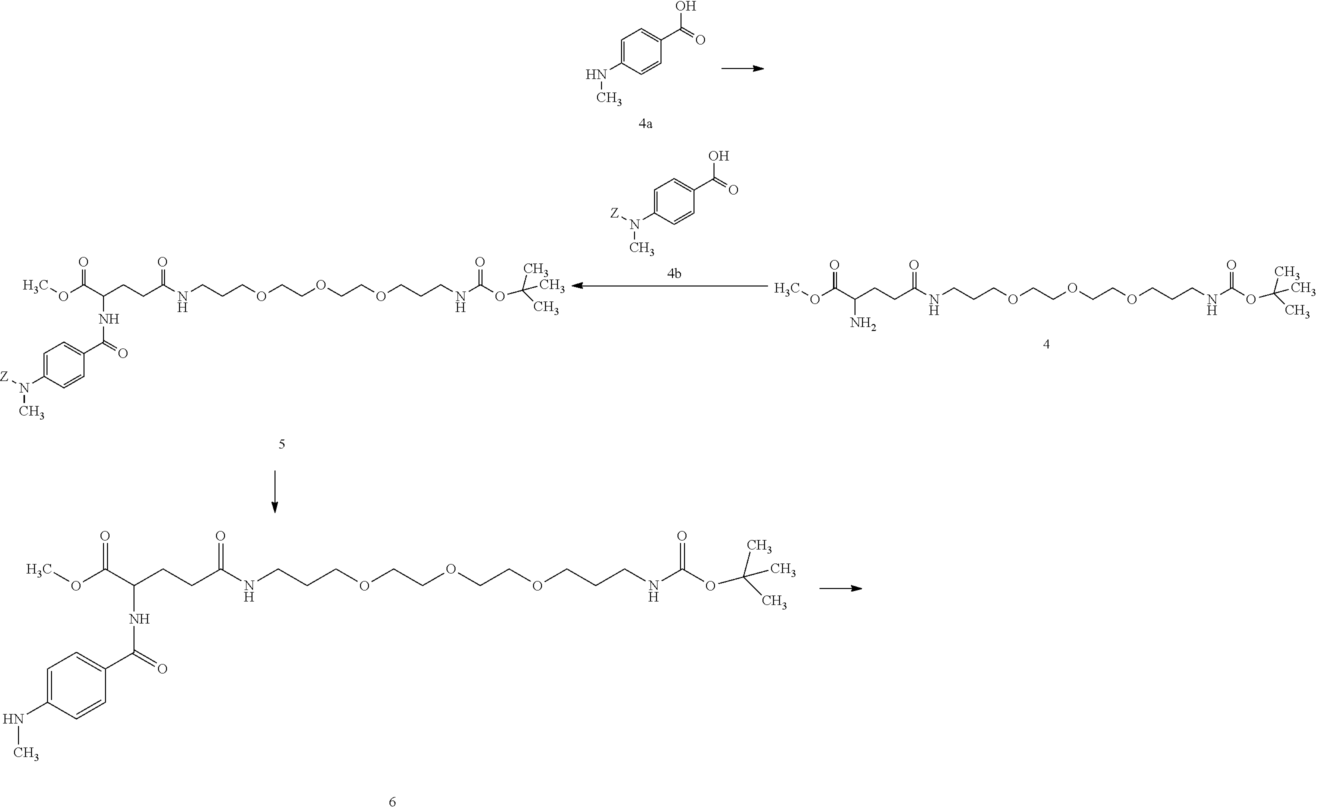

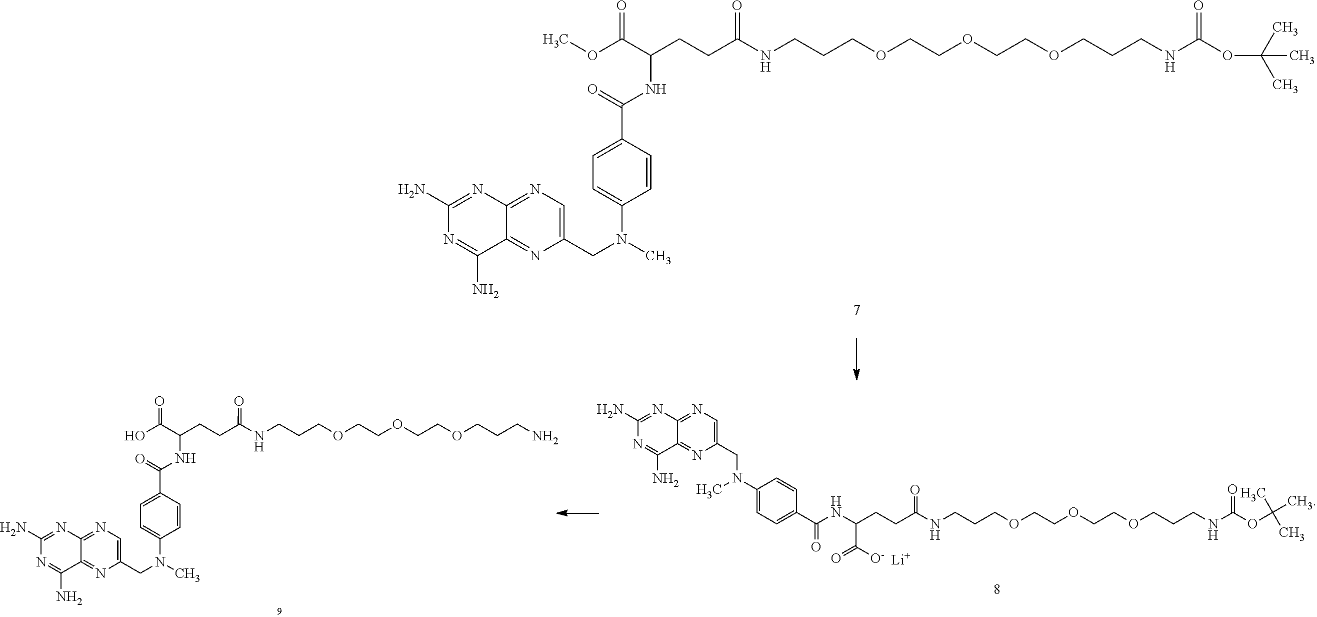 [SMILES: COC(=O)C(CCC(=O)NCCCOCCOCCOCCCNC(=O)OC(C)(C)C)NC(=O)c1ccc(N(C)Cc2cnc3nc(N)nc(N)c3n2)cc1 ; CN(Cc1cnc2nc(N)nc(N)c2n1)c1ccc(C(=O)NC(CCC(=O)NCCCOCCOCCOCCCN)C(=O)O)cc1 ; CN(Cc1cnc2nc(N)nc(N)c2n1)c1ccc(C(=O)NC(CCC(=O)NCCCOCCOCCOCCCNC(=O)OC(C)(C)C)C(=O)[O-])cc1 ; [Li+]]

In an eleventh aspect the present invention provides a process for the production of a nanoparticle of the first aspect of the invention, comprising:
(a) providing a compound of the formula D-L₁-NH₂, wherein D comprises an antifolate drug or folic acid, L₁ comprises —(OCH₂CH₂)ₚ—, wherein p is an integer in the range 1 to 10;
(b) providing a nanoparticle comprising:
   a core comprising a metal and/or a semiconductor; and
   a plurality of ligands covalently linked to the core, wherein said ligands comprise:
(i) at least one dilution ligand comprising a carbohydrate, glutathione or an ethyleneglycol-containing moiety; and
(ii) a ligand of the formula COOH-L₂-S, wherein L₂ comprises —(OCH₂CH₂)_{q}—, wherein q is an integer in the range 1 to 10, and wherein the terminal sulfur atom of said ligand is covalently bound to said core; and
(c) reacting the compound of formula D-L₁-NH₂ with the nanoparticle under conditions which allow an amide bond to form between the terminal amine group of the compound of formula D-L₁-NH₂ and the carboxylic acid group of the nanoparticle ligand of the formula COOH-L₂-S.

In some embodiments D comprises the following structure:

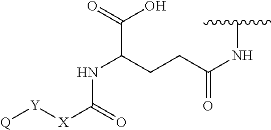

wherein;
   X is a 3 to 8 membered (e.g. 5 or 6 membered) carbocyclic, heterocyclic, carboaromatic or heteroaromatic ring,
   Y is a linker group having 1 to 20 atoms comprising one or more atoms selected from H, C, N, O and S;
   wherein Y is optionally substituted by one or more groups having 1 to 20 atoms comprising of one or more atoms selected from H, C, N, O and S, and
   Q is a fused bicyclic heterocyclic or heteroaromatic ring optionally substituted with one or more groups selected from amino, hydroxyl, carbonyl, methyl, ethyl, propyl, isopropyl, butyl and isobutyl.

In particular, D may comprise an antifolate drug selected from the group consisting of: methotrexate, pemetrexed, ralitrexed and pralatrexate, or may comprise folic acid.

In some embodiments D is selected from the following structures:

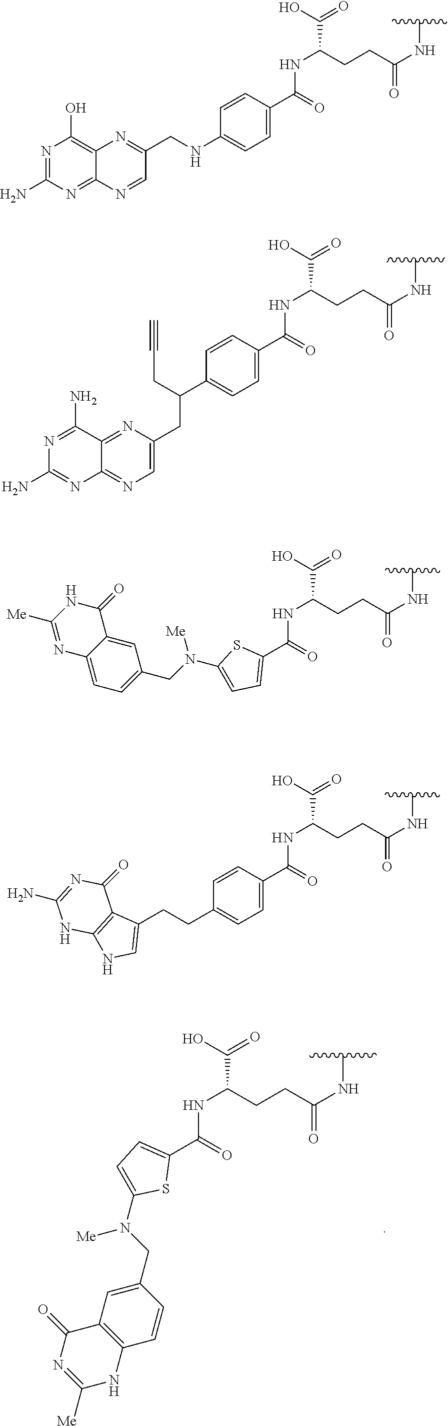 [SMILES: *NC(=O)CC[C@H](NC(=O)c1ccc(NCc2cnc3nc(N)nc(O)c3n2)cc1)C(=O)O ; *NC(=O)CC[C@H](NC(=O)c1ccc(C(CC#C)Cc2cnc3nc(N)nc(N)c3n2)cc1)C(=O)O ; *NC(=O)CC[C@H](NC(=O)c1ccc(N(C)Cc2ccc3nc(C)[nH]c(=O)c3c2)s1)C(=O)O ; *NC(=O)CC[C@H](NC(=O)c1ccc(CCc2c[nH]c3[nH]c(N)nc(=O)c23)cc1)C(=O)O ; *NC(=O)CC[C@H](NC(=O)c1ccc(N(C)Cc2ccc3[nH]c(C)nc(=O)c3c2)s1)C(=O)O]

In some embodiments the process comprises:

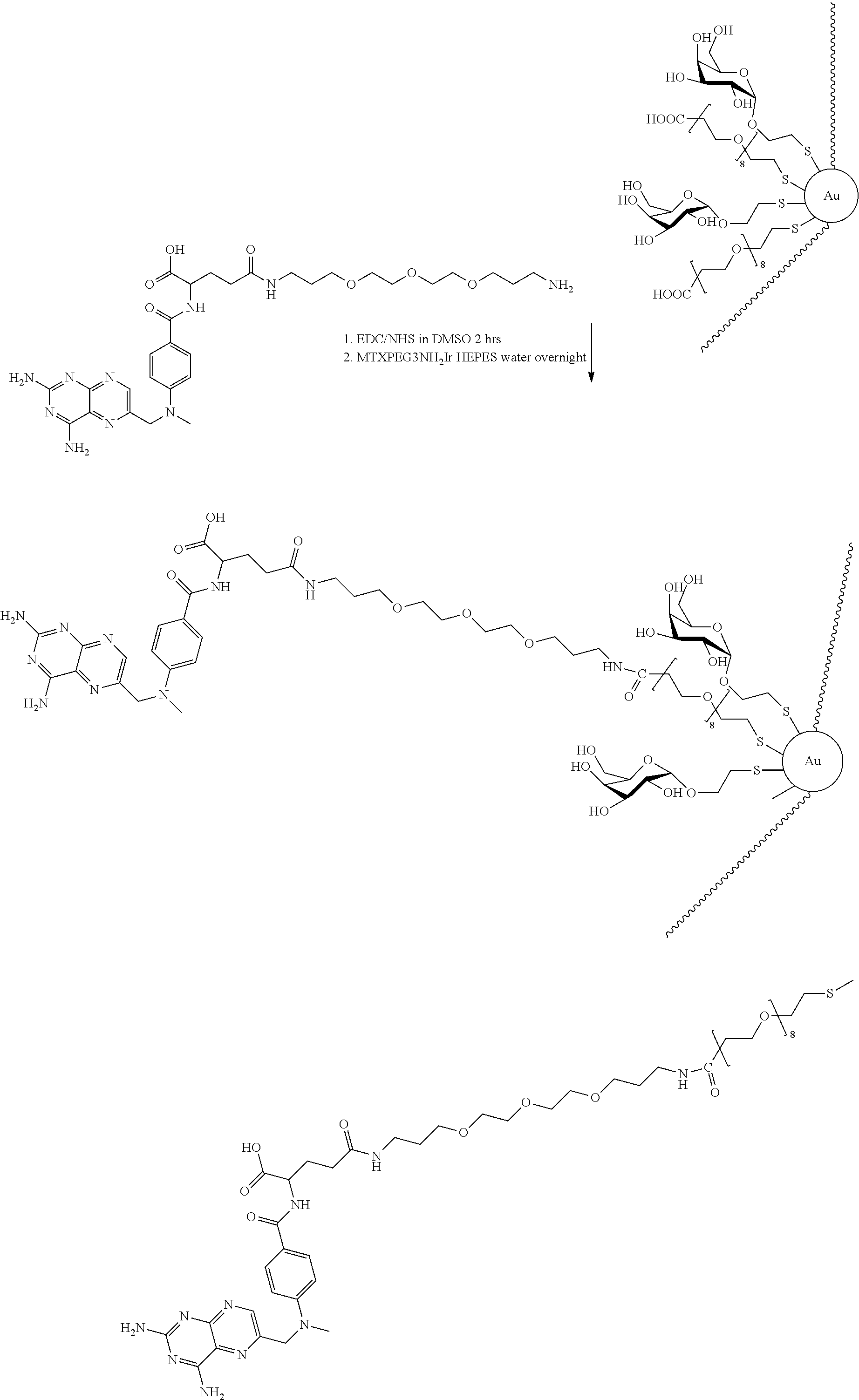

In a twelfth aspect, the invention provides a process for forming a nanoparticle of claim 1 comprising a step of mixing;
a payload-free nanoparticle having an L₂ ligand and at least one dilution ligand comprising a carbohydrate, glutathione or an ethylene glycol-containing moiety; and
a free D-L₁ ligand,
wherein D, L₁, Z and L₂ are as defined in claim 1 and one of the L₂ ligand and the D-L₁ ligand has a terminal alkyne group and the other has a terminal azide group
such that a nanoparticle is formed having a D-L₁-Z-L₂ ligand wherein Z is a 1,2,3-triazole.

In some embodiments the step of mixing is;

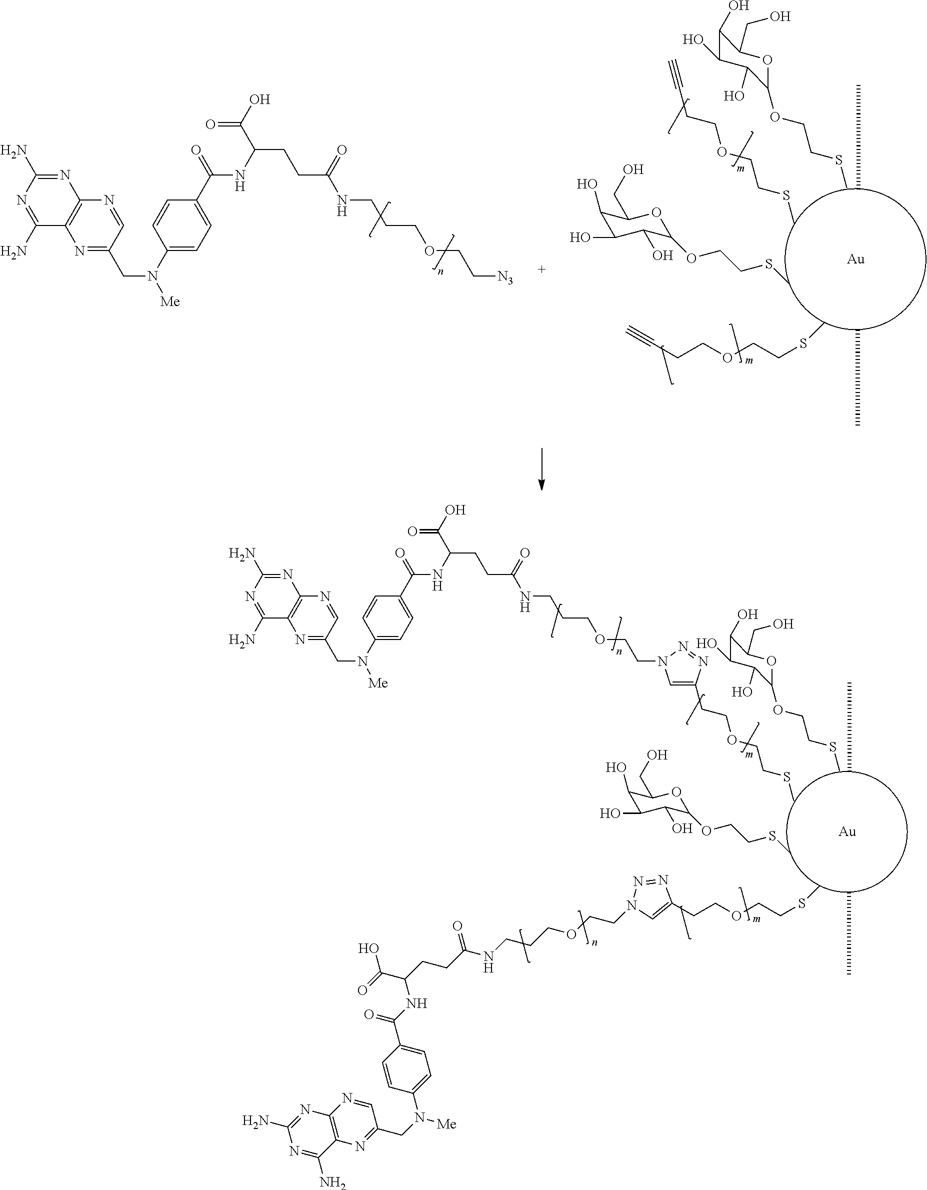

wherein n and m are independently 1, 2, 3, 4, 5, 6, 7, 8, 9 or 10, the total number of ligands per core is at least 20, and the total number of methotrexate-containing ligands per core is at least 10.

In some embodiments the step of mixing is;

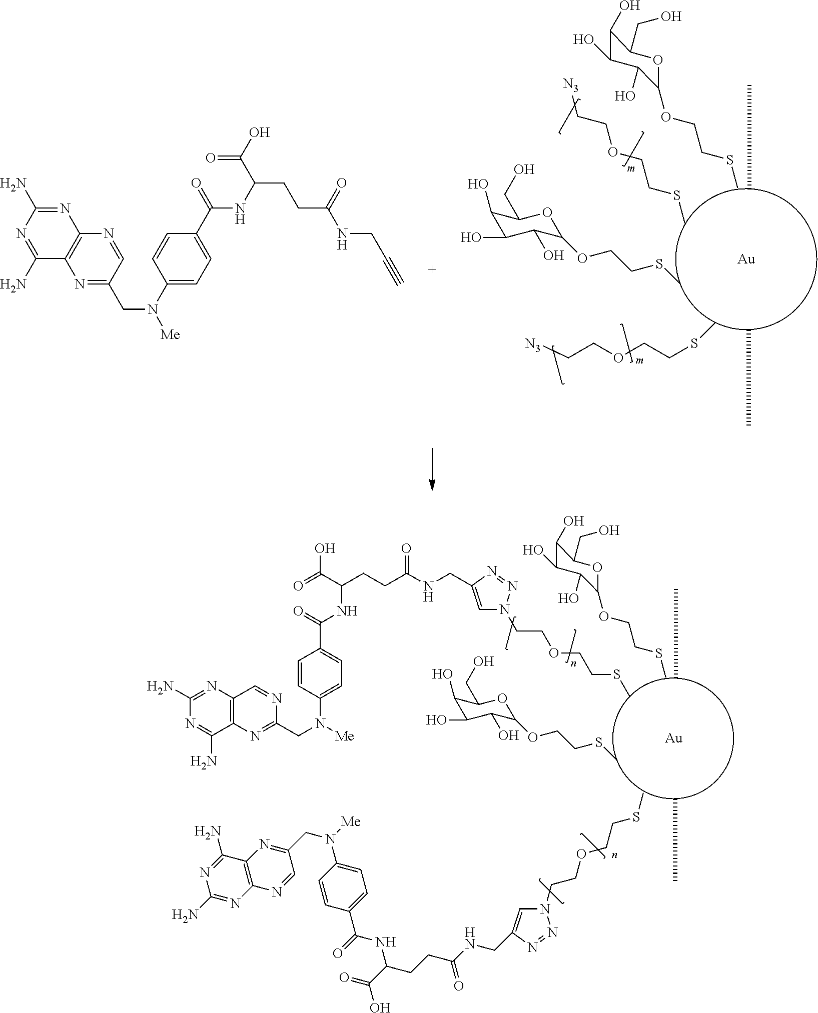

wherein n is an integer of between 1 and 15, the total number of ligands per core is at least 5, and the total number of methotrexate-containing ligands per core is at least 3.

In a thirteenth aspect, the invention provides a process for forming a nanoparticle according to the first aspect comprising a step of mixing a ligand of the formula D-L₁-Z-L₂ with a payload-free nanoparticle;
wherein the payload-free nanoparticle has a core comprising a metal and/or a semiconductor that is covalently bound to a plurality of dilution ligands having a carbohydrate, glutathione or an ethylene glycol-containing moiety;
such that some of the dilution ligands are displaced by the ligands.

It may be that the ligand of the formula D-L₁-Z-L₂ is either D-L₁-Z-L₂-SH (a free thiol) or D-L₁-Z-L₂-S—S-L₂-Z-L₁-D (a disulfide) that is reduced in situ.

In some embodiments the dilution ligands are covalently bound to the payload-free nanoparticle via a sulfur atom.

In accordance with any aspect of the present invention, the subject may be a human, a companion animal (e.g. a dog or cat), a laboratory animal (e.g. a mouse, rat, rabbit, pig or non-human primate), a domestic or farm animal (e.g. a pig, cow, horse or sheep). Preferably, the subject is a human who has been diagnosed as having a proliferative disorder (e.g. a cancer), an inflammatory disorder or an autoimmune disease.

Embodiments of the present invention will now be described by way of example and not limitation with reference to the accompanying figures. However various further aspects and embodiments of the present invention will be apparent to those skilled in the art in view of the present disclosure.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1 depicts the general chemical structure of a gold core nanoparticle having a corona comprising alpha-galactose-C2-SH ligands and MTX-PEG₃NHC(O)PEG₈-SH ligands, also described herein as MTX-PEG₃-NH₂-loaded GNPs.

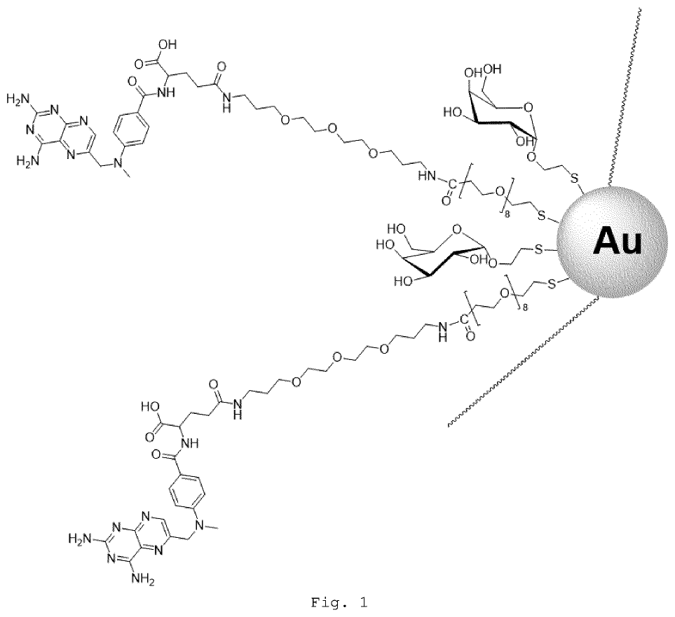

FIG. 2 shows a ¹H NMR spectrum confirming the chemical structure of 4-[(3-{2-[2-(3-aminopropoxy)ethoxy]ethoxy}propyl)carbamoyl]-2-[(4-{[(2,4diaminopteridin6yl)methyl](methyl)amino}phenyl)formamido]butanoic acid. Peaks are identified and the chemical structure is shown as an insert.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

In describing the present invention, the following terms will be employed, and are intended to be defined as indicated below.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### Nanoparticles

As used herein, “nanoparticle” refers to a particle having a nanomeric scale, and is not intended to convey any specific shape limitation. In particular, “nanoparticle” encompasses nanospheres, nanotubes, nanoboxes, nanoclusters, nanorods and the like. In certain embodiments the nanoparticles and/or nanoparticle cores contemplated herein have a generally polyhedral or spherical geometry. References to “diameter” of a nanoparticle or a nanoparticle core a generally taken to mean the longest dimension of the nanoparticle or nanoparticle core, respectively. For nanoparticles having a substantially polyhedral or spherical geometry, the shortest dimension across the particle will typically be within 50% of the longest dimension across the particle and may be, e.g., within 25% or 10%.

Nanoparticles comprising a plurality of carbohydrate-containing ligands have been described in, for example, WO 2002/032404, WO 2004/108165, WO 2005/116226, WO 2006/037979, WO 2007/015105, WO 2007/122388, WO 2005/091704 (the entire contents of each of which is expressly incorporated herein by reference) and such nanoparticles may find use in accordance with the present invention.

As used herein, “corona” refers to a layer or coating, which may partially or completely cover the exposed surface of the nanoparticle core. The corona includes a plurality of ligands covalently attached to the core of the nanoparticle. Thus, the corona may be considered to be an organic layer that surrounds or partially surrounds the metallic core. In certain embodiments the corona provides and/or participates in passivating the core of the nanoparticle. Thus, in certain cases the corona may include a sufficiently complete coating layer substantially to stabilise the core. In certain cases the corona facilitates solubility, such as water solubility, of the nanoparticles of the present invention.

Nanoparticles are small particles, e.g. clusters of metal or semiconductor atoms, that can be used as a substrate for immobilising ligands.

Preferably, the nanoparticles have cores having mean diameters between 0.5 and 50 nm, more preferably between 0.5 and 10 nm, more preferably between 0.5 and 5 nm, more preferably between 0.5 and 3 nm and still more preferably between 0.5 and 2.5 nm. When the ligands are considered in addition to the cores, preferably the overall mean diameter of the particles is between 2.0 and 50 nm, more preferably between 3 and 10 nm and most preferably between 4 and 5 nm. The mean diameter can be measured using techniques well known in the art such as transmission electron microscopy.

The core material can be a metal or semiconductor and may be formed of more than one type of atom. Preferably, the core material is a metal selected from Au, Fe or Cu. Nanoparticle cores may also be formed from alloys including Au/Fe, Au/Cu, Au/Gd, Au/Fe/Cu, Au/Fe/Gd and Au/Fe/Cu/Gd, and may be used in the present invention. Preferred core materials are Au and Fe, with the most preferred material being Au. The cores of the nanoparticles preferably comprise between about 100 and 500 atoms or 100 and 2,000 (e.g. gold atoms) to provide core diameters in the nanometre range. Other particularly useful core materials are doped with one or more atoms that are NMR active, allowing the nanoparticles to be detected using NMR, both in vitro and in vivo. Examples of NMR active atoms include Mn⁺², Gd⁺³, Eu⁺², Cu⁺², V⁺², Co⁺², Ni⁺², Fe⁺², Fe⁺³ and lanthanides⁺³, or the quantum dots.

Nanoparticle cores comprising semiconductor compounds can be detected as nanometre scale semiconductor crystals are capable of acting as quantum dots, that is they can absorb light thereby exciting electrons in the materials to higher energy levels, subsequently releasing photons of light at frequencies characteristic of the material. An example of a semiconductor core material is cadmium selenide, cadmium sulphide, cadmium telluride. Also included are the zinc compounds such as zinc sulphide.

In some embodiments, the nanoparticle or its ligand comprises a detectable label. The label may be an element of the core of the nanoparticle or the ligand. The label may be detectable because of an intrinsic property of that element of the nanoparticle or by being linked, conjugated or associated with a further moiety that is detectable.

### Antifolate Drug

As used herein “antifolate” or “antifolate drug” refers to members of the class of antifolates that antagonise the actions of folic acid. Antifolates specifically contemplated herein include: methotrexate, pemetrexed, ralitrexed and pralatrexate. In their free form, the antifolate drugs typically have a terminal carboxylic acid that is useful to, for example, undergo an amide coupling.

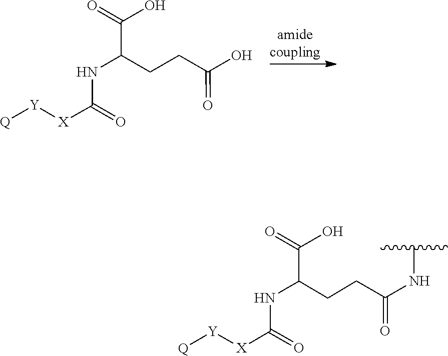

In some embodiments the antifolate drug may comprise the following structure:

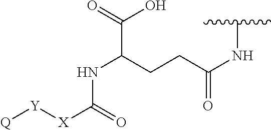

wherein;
X is a 3 to 8 membered (e.g. 5 or 6 membered) carbocyclic, heterocyclic, carboaromatic or heteroaromatic ring,
Y is a linker group having 1 to 20 atoms comprising one or more atoms selected from H, C, N, O and S;
wherein Y is optionally substituted by one or more groups having 1 to 20 atoms comprising of one or more atoms selected from H, C, N, O and S, and
Q is a fused bicyclic heterocyclic or heteroaromatic ring optionally substituted with one or more groups selected from amino, hydroxyl, carbonyl, methyl, ethyl, propyl, isopropyl, butyl and isobutyl. In particular, Q may be a substituted pteridine.

In some embodiments, for example, for use in applications which target the folate receptor, D may comprise folic acid.

In some embodiments D may be selected from the following structures:

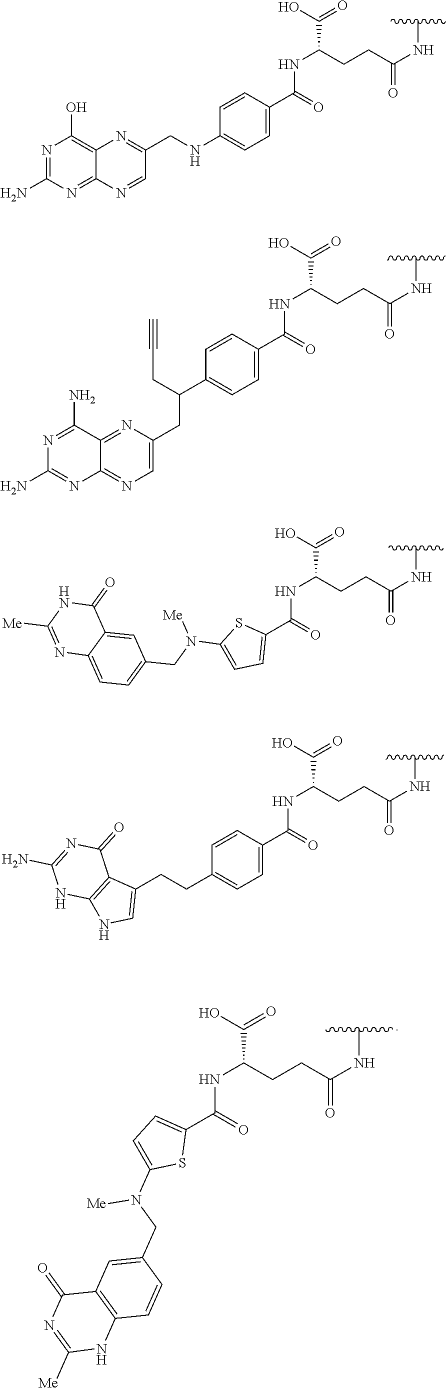 [SMILES: *NC(=O)CC[C@H](NC(=O)c1ccc(NCc2cnc3nc(N)nc(O)c3n2)cc1)C(=O)O ; *NC(=O)CC[C@H](NC(=O)c1ccc(C(CC#C)Cc2cnc3nc(N)nc(N)c3n2)cc1)C(=O)O ; *NC(=O)CC[C@H](NC(=O)c1ccc(N(C)Cc2ccc3nc(C)[nH]c(=O)c3c2)s1)C(=O)O ; *NC(=O)CC[C@H](NC(=O)c1ccc(CCc2c[nH]c3[nH]c(N)nc(=O)c23)cc1)C(=O)O ; *NC(=O)CC[C@H](NC(=O)c1ccc(N(C)Cc2ccc3[nH]c(C)nc(=O)c3c2)s1)C(=O)O]

### Methotrexate

Methotrexate (MTX), formerly known as amethopterin, is a chemotherapy agent and immune system suppressant. It has found use in the treatment of various cancers, autoimmune diseases, ectopic pregnancy, and for medical abortions.

MTX has the CAS number 59-05-2 and has the structure depicted below:

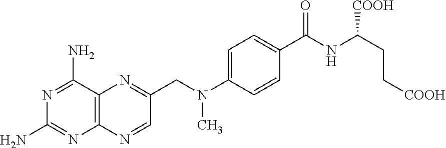 [SMILES: CN(Cc1cnc2nc(N)nc(N)c2n1)c1ccc(C(=O)N[C@@H](CCC(=O)O)C(=O)O)cc1]

As used herein “methotrexate” or “MTX” refers to not only the compound of the of the above chemical formula, but also derivatives of MTX in which one or more functional groups have been modified for attachment to the nanoparticle via the linker L. In particular, MTX may be bonded to linker L via, e.g., an amide formed at a carboxylic acid group in the above structure (particularly the carboxylic acid group that is the further from the amide bond of the two carboxylic acid groups shown in the structure above).

### Folate Receptor

“Folate receptor” specifically includes the human folate receptor alpha (UniProt accession number P15328, 1 Jun. 1994—v3, checksum D458D8BB047C96A6, the contents of which is incorporated herein by reference), human folate receptor beta (UniProt accession number P14207, 17 Oct. 2006—v4, checksum F585715CF5631C98) and human folate receptor gamma (UniProt accession number P41439, 1 Nov. 1995—v1, checksum AC7636EB5355647B). A “folate receptor binding agent” or “folate receptor targeting agent” refers to a compound, which may be an antifolate or folic acid, that binds to a folate receptor. In some cases, the folate receptor binding agent may be capable of being bound by the folate receptor and transported into a cell via endocytosis. In some cases a folate receptor binding agent may be folic acid or an antifolate (for example methotrexate or a linker-coupled methotrexate such as MTX-(EG)₃-NH₂).

### Ethylene Glycol

As used herein, an ethylene glycol-containing linker or chain means that one or more ethylene glycol subunits is present. This may be depicted or represented in a variety of ways, such as —(OCH₂CH₂)ₘ— or (EG)ₙ or (PEG)ₘ or PEGₘ or PEGm, where m is a number. Unless context dictates otherwise, these terms are used interchangeably herein. Thus, the term “PEG” may be employed herein to mean shorter, e.g., oligomer length chains of ethylene glycol units, such as PEG3 or PEG8, which have the same meaning as (EG)₃ and (EG)₈, respectively.

### Gel

A gel is a non-fluid colloidal network or polymer network that is expanded throughout its volume by a fluid. In the present context, the gel may be a pharmaceutically acceptable gel, e.g., a hydrogel. A particularly suitable class of hydrogels are hydrogels formed of the Carbopol® family of crosslinked polyacrylic acid polymers available from Lubrizol Corporation and described at https://www.lubrizol.com/Life-Sciences/Products/Carbopol-Polymer-Products

### Administration and Treatment

The nanoparticles and compositions of the invention may be administered to patients by any number of different routes, including enteral or parenteral routes. Parenteral administration includes administration by the following routes: intravenous, cutaneous or subcutaneous, nasal, intramuscular, intraocular, transepithelial, intraperitoneal and topical (including dermal, ocular, rectal, nasal, inhalation and aerosol), and rectal systemic routes. A preferred route of administration is dermal administration by topical application to the skin.

The nanoparticles of the invention may be formulated as pharmaceutical compositions that may be in the forms of solid or liquid compositions. Such compositions will generally comprise a carrier of some sort, for example a solid carrier or a liquid carrier such as water, petroleum, animal or vegetable oils, mineral oil or synthetic oil. Physiological saline solution, or glycols such as ethylene glycol, propylene glycol or polyethylene glycol may be included. Such compositions and preparations generally contain at least 0.1 wt % of the compound.

For intravenous, cutaneous or subcutaneous injection, or injection at the site of affliction, the active ingredient will be in the form of a parenterally acceptable aqueous solution or liquid which is pyrogen-free and has suitable pH, tonicity and stability. Those of relevant skill in the art are well able to prepare suitable solutions using, for example, solutions of the compounds or a derivative thereof, e.g. in physiological saline, a dispersion prepared with glycerol, liquid polyethylene glycol or oils.

In addition to one or more of the compounds, optionally in combination with another active ingredient, the compositions can comprise one or more of a pharmaceutically acceptable excipient, carrier, buffer, stabiliser, isotonicising agent, preservative or anti-oxidant or other materials well known to those skilled in the art. Such materials should be non-toxic and should not interfere with the efficacy of the active ingredient. The precise nature of the carrier or other material may depend on the route of administration, e.g., topical application or intravenous injection.

Preferably, the pharmaceutically compositions are given to an individual in a prophylactically effective amount or a therapeutically effective amount (as the case may be, although prophylaxis may be considered therapy), this being sufficient to show benefit to the individual. Typically, this will be to cause a therapeutically useful activity providing benefit to the individual. The actual amount of the compounds administered, and rate and time-course of administration, will depend on the nature and severity of the condition being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Handbook of Pharmaceutical Additives, 2nd Edition (eds. M. Ash and I. Ash), 2001 (Synapse Information Resources, Inc., Endicott, N.Y., USA); Remington's Pharmaceutical Sciences, 20th Edition, 2000, pub. Lippincott, Williams & Wilkins; and Handbook of Pharmaceutical Excipients, 2nd edition, 1994. By way of example, the compositions are preferably administered to patients in dosages of between about 0.01 and 100 mg of active compound per kg of body weight, and more preferably between about 0.5 and 10 mg/kg of body weight. In the context of treatment of a skin disorder, one benefit of topical administration of a composition of the present invention is that the resulting systemic concentration of methotrexate will be significantly lower than if methotrexate were administered systemically. This means that toxic and other unwanted side effects of methotrexate can be minimised or substantially avoided while nevertheless achieving clinically beneficial concentrations of methotrexate at the affected site(s) of the subject's skin.

The following is presented by way of example and is not to be construed as a limitation to the scope of the claims.

### EXAMPLES

### Comparative Example 1—Synthesis of Methotrexate-Coupled Gold Nanoparticles (MTX-GNPs)

Preparation of Ligands and Synthesis of [α-Gal]₂₂[AL]₂₂@Au GNPs

Gold nanoparticles having a corona of alpha-galactose-C2 (α-Gal) and 1-amino-6-mercapto-hexaethylenglycol (SH—CH₂-(EG)₆-NH₂ also known as “amino linker” or “AL”) ligands were synthesised as described previously (see WO2011/154711, Examples 1 and 2, and WO2016/102613, Example 1, both of which documents are incorporated herein by reference).

### Preparation of 2-thio-ethyl-α-D-galactoside (α-galactose-C2SH “α-Gal”)

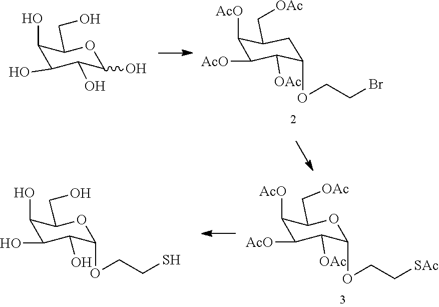

To a suspension of galactose (3 g, 16.65 mmol) in 2-bromoethanol (30 ml), acid resin Amberlite 120-H is added to reach pH 2. The reaction is stirred for 16 hours at 50-60° C. The reaction mixture is filtered and washed with MeOH. Triethylamine is added to reach pH 8. The crude of the reaction is concentrated and co evaporated 3 times with toluene. The reaction mixture is dissolved pyridine (75 mL) and Ac2O (35 mL) and a catalytic amount of DMAP are added at 0° C. and stirred for 3h at rt. The mixture is diluted with AcOEt and washed with 1.H₂O; 2.HCl (10%) 3. NaHCO₃ dis 4. H₂O. The organic layer is collected and dried over anhydrous Na₂SO₄. TLC (Hexane: AcOEt 3:1, 2 elutions) shows a major product (desired) and a lower Rf minority. The product is purified by flash chromatography using the mixture hexane:ethyl acetate 6:1 as eluent and the 2-bromoethyl-alpha-galactoside (2) is obtained.

The product of the previous reaction, 2 is dissolved in 27 ml of 2-butanone. To this solution, a catalytic amount of tetrabutylammonium iodide and 4 equivalents of potassium thioacetate are added. The resulting suspension is stirred for 2 hours at room temperature. Throughout this period the reaction is tested by TLC (hexane-AcOEt 2:1, 2 elutions) for the disappearance of the starting material. The mixture is diluted with 20 ml of AcOEt and washed with a saturated NaCl solution. The organic phase is dried, filtered and evaporated under vacuum. The product is purified in hexane/AcOEt 2:1→1:1 to obtain the acetylthio-alpha-galactoside 3.

The new product of the reaction, 3 is dissolved in a mixture dichloromethane-methanol 2:1. To this mixture a solution of 1N sodium methoxide (1 equivalent) is added and stirred for 1 hour at room temperature. Amberlite IR-120H resin is added to achieve pH 5-6. The resulting mixture is then filtered and concentrated to dryness to obtain the final product (α-galactose C2SH).

### Preparation of Amino-Thiol Linker (AL)

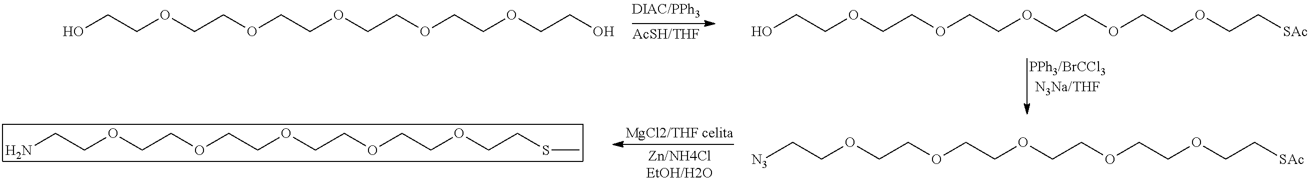

To a solution of PPh₃ (3 g, 11.4 mmol) in 20 ml dry THF, DIAC (2.3 g, 11.4 mmol) is added. The mixture is allowed to stir at 0° C. 15 min until the appearance of a white product. To this mixture a solution of hexaethyleneglycol (1.45 mL, 5.7 mmol) and HSAc (610 μl, 8.55 mmol) in dry THF (20 mL) is added dropwise (addition funnel). After 15 min the products begin to appear on TLC at Rf 0.2. The solution is concentrated in an evaporator. The crude of the reaction is dissolved in 50 ml of dichloromethane and washed with a solution of K₂CO₃ 10%. The organic phase is dried over anhydrous Na₂SO₄, filtered and concentrated under vacuum. Flash chromatography of the crude using AcOEt: Hexane 1:1, AcOEt and finally DCM:MeOH 4:1 as eluent gave the acetyl-thio-hexaethyleneglycol derivative.

The reaction product is dissolved in 5 ml of DMF and PPh₃ (2.25 g, 8.55 mmol), NaN₃ (0.741 g, 11.4 mmol) and BrCl₃C (0,845 ml, 8.55 mmol) are added and the solution subsequently stirred for 40 min at room temperature. The resulting product has a higher Rf than the starting product when performing TLC (DCM:MeOH 25:1). The reaction mixture is diluted with 100 ml of diethylether and washed three times with H₂O. The organic phase is dried over anhydrous Na₂SO₄, filtered and evaporated under vacuum. The product is purified by flash chromatography using the mixture of eluents DMC/MeOH 200:1 and DCM/MeOH 40:1 to obtain the azido-acetylthio-hexaethyleneglycol derivative.

To remove the triphenyl phosphine oxide, the reaction product is dissolved in 10 ml of THF and 0.5 g of MgCl₂ is added to this solution. The reaction is stirred for 2h at 80° C. until a white precipitate appears and then is filtered through celite. The product is dissolved in a mixture of ethanol:H₂O 3:1 and added Zn dust (0.45 g, 6.84 mmol) and NH₄C1 (0.6 g, 11.4 mmol). The reaction was stirred at reflux for 1h until the presence of starting material is no longer detectable by TLC (DCM/MeOH 25:1). The reaction is filtered through celite and the solvent is evaporated. The crude reaction product is diluted with AcOEt and extracted with 5 ml H₂O. The aqueous phase is evaporated to dryness to obtain the amino-thiol-hexaethylenglycol product.

### Synthesis of [α-Gal]22[AL]22@Au GNPs

Alpha-galactose C2 derivative 3 and hexaethyleneglycol amine linker 6 were taken from Midatech Biogune stock. N-(3-Dimethylaminopropyl)-N′-ethylcarbodiimide hydrochloride (EDC-HCl), HAuCl₄, NaBH₄ were purchased from Sigma-Aldrich Chemical Company. Imidazole-4-acetic acid monohydrochloride was purchased from Alfa Aesar. Company High quality MeOH and Nanopure water (18.1 mΩ) were used for all experiments and solutions.

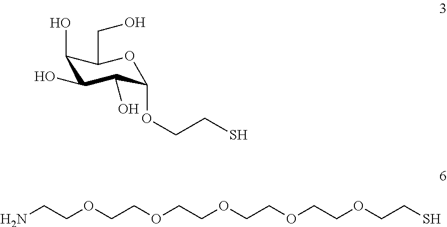

To a mix of amine-mercapto hexaethylenglycol linker 6 and alpha-galactose ligand 3 in a ratio 1:1 (0.58 mmol, 3 eq.) in MeOH (49 mL) was added an aqueous solution of gold salt (7.86 mL, 0.19 mmol, 0.025M). The reaction was stirred for 30 seconds and then, an aqueous solution of NaBH₄ (1N) was added in several portions (4.32 mL, 4.32 mmol). The reaction was shaken for 100 minutes at 900 rpm. After this time, the suspension was centrifuged 1 minute at 14000 rpm. The supernatant is removed and the precipitated was dissolved in 2 mL of water. Then, 2 mL of the suspension were introduced in two filters (Amicon, 10 KDa, 4 mL) and were centrifuged 5 minutes at 4500 g. The residue in the filter was washed twice more with water. The final residue was dissolved in 80 mL of water.

Functionalisation of [α-Gal]₂₂[AL]22@Au GNPs with Methotrexate

Functionalisation of the [α-Gal]₂₂[AL]₂₂@Au nanoparticles prepared as described above with methotrexate was performed using 1-Ethyl-3-(3-dimethylaminopropyl)carbodiimide (EDC) and N-hydroxysuccinimide (NHS) in dimethyl sulfoxide (DMSO) at room temperature according to the following scheme:

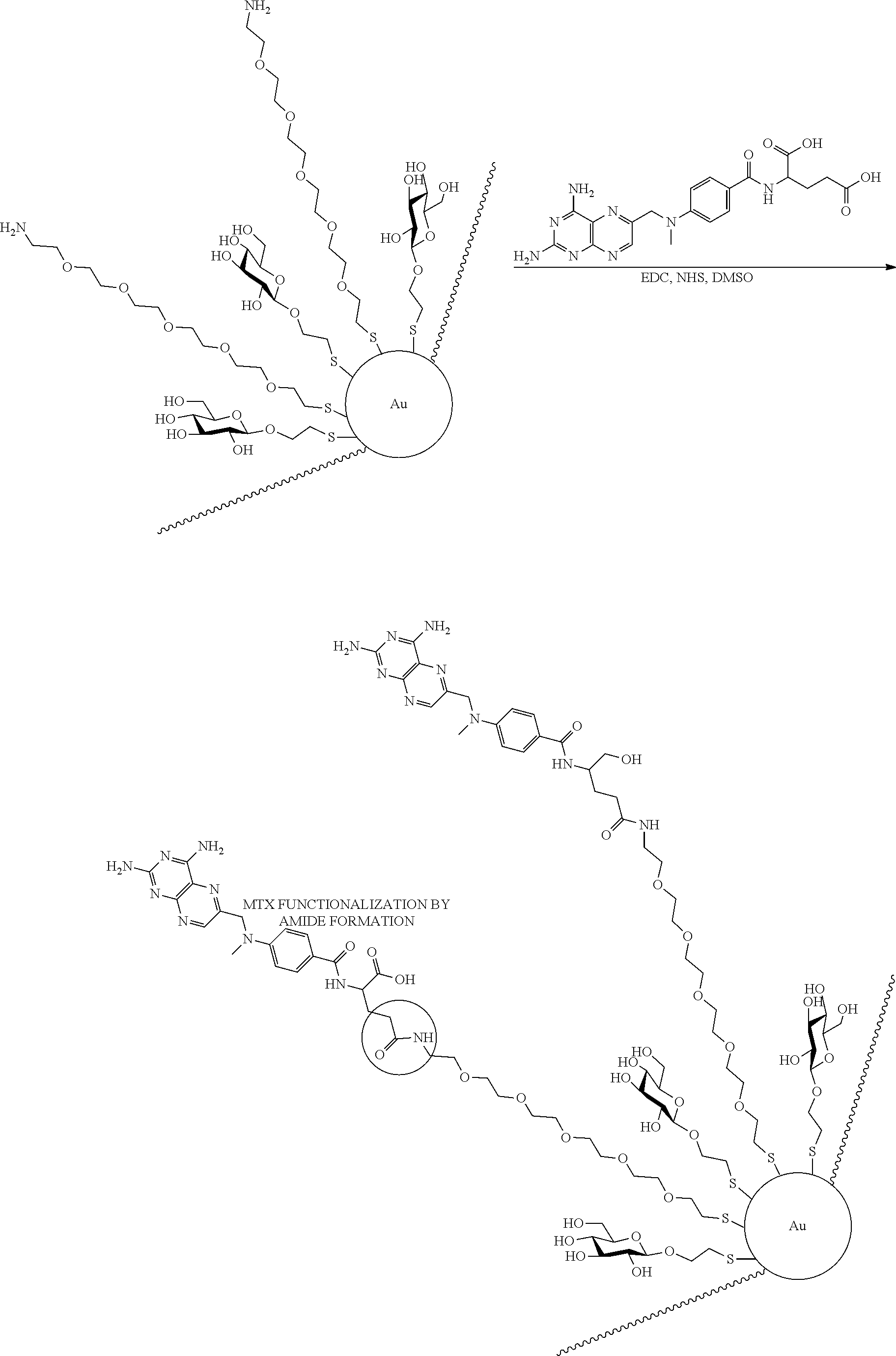

### Materials

| Material | Supplier | Batch No. |
|---|---|---|
| Nanoparticle | Midatech Pharma | M199-082 |
| EDC | SIGMA-ALDRICH | S2BK8745V |
| NHS | ALDRICH | MKBP79891V |
| MTX | AVACHEM | ZW0701 |
| DMSO | SIGMA-ALDRICH | SHB61596V |

### Procedure

The nanoparticles were concentrated by centrifugation and collected with DMSO (3.62 mL) to obtain about 8000 ppm of gold concentration.

### Drug Activation

To a solution of MTX (0.1M) in DMSO, EDC (38.4 μL; 0.5M) was added and the mixture was stirred about five minutes. Then, NHS (19.2 μL; 1.0M) was added and the mixture was activated for thirty minutes at room temperature.

### Drug Functionalization

[α-Gal]₂₂[AL]₂₂@Au GNPs (750 μL) were added to the previously activated solution and the coupling was incubated overnight at room temperature in darkness.

### Purification

The nanoparticles were purified by centrifugation (4500 rpm, 10 min) using NaOH 0.1M as eluent. The content was collected in 500 μL H₂O (12.00 μg/μL) and was stored for further analysis.

### Analysis

Gold content was assessed by inductively coupled plasma mass spectrometry (ICP-MS), size by dynamic light scattering (DLS) electrostatic charge by zeta potential, and structure by ¹H NMR.

DLS size indicated a main peak at 5.15 nm. However, a secondary peak at 1.61 nm was also observed indicating two populations of nanoparticles. Differential centrifugation sedimentation (DCS) analysis confirmed the presence of two populations of nanoparticles, with sizes of 3.0 nm and 8.0 nm.

Zeta potential was found to be −51.1 mV (i.e. negatively charged).

The above procedure was repeated with different equivalents of MTX. In each case the final loading of MTX per nanoparticle was determined by ¹H NMR analysis. MTX loadings from 2 equivalents/GNP up to ˜5 equivalents/GNP were obtained.

### CONCLUSIONS

The above results demonstrate successful synthesis of [α-Gal]-[MTX-AL]@Au GNPs with size <10 nm and up to 5 equivalents of MTX per GNP. However, variability was observed between batches for GNP size and zeta potential. Methotrexate has two potential carboxylate binding sites that may lead to variability in binding capacity to the amine groups on positively charged GNPs (i.e. possible dual EDC activation of MTX may explain a heterogeneous product).

### Example 2—Synthesis of Modified Methotrexate

The present inventors aimed to increase the MTX loading per GNP and to reduce variability due to the multiple carboxyl groups on MTX observed in Example 1.

To this end, a modified methotrexate having a (EG)₃NH₂ linker was synthesised.

### 4-[(3-{2-[2-(3-aminopropoxy)ethoxy]ethoxy}propyl)carbamoyl]-2-[(4-{[(2,4diaminopteridin6yl)methyl](methyl)amino}phenyl)formamido]butanoic Acid

### Structural Formula:

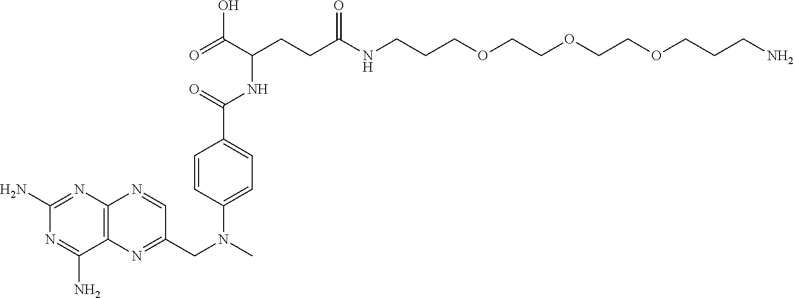 [SMILES: CN(Cc1cnc2nc(N)nc(N)c2n1)c1ccc(C(=O)NC(CCC(=O)NCCCOCCOCCOCCCN)C(=O)O)cc1]

### Molecular Formula:

C₃₀H₄₄N₁₀O₇

### Molecular Weight:

656.75 g/mol

### Synthesis of 4[(3{2[2(3aminopropoxy)ethoxy]ethoxy}propyl)carbamoyl]2[(4{[(2,4-diaminopteridin-6-yl)methyl](methyl)amino}phenyl)formamido]butanoic Acid was in Accordance with Scheme 1

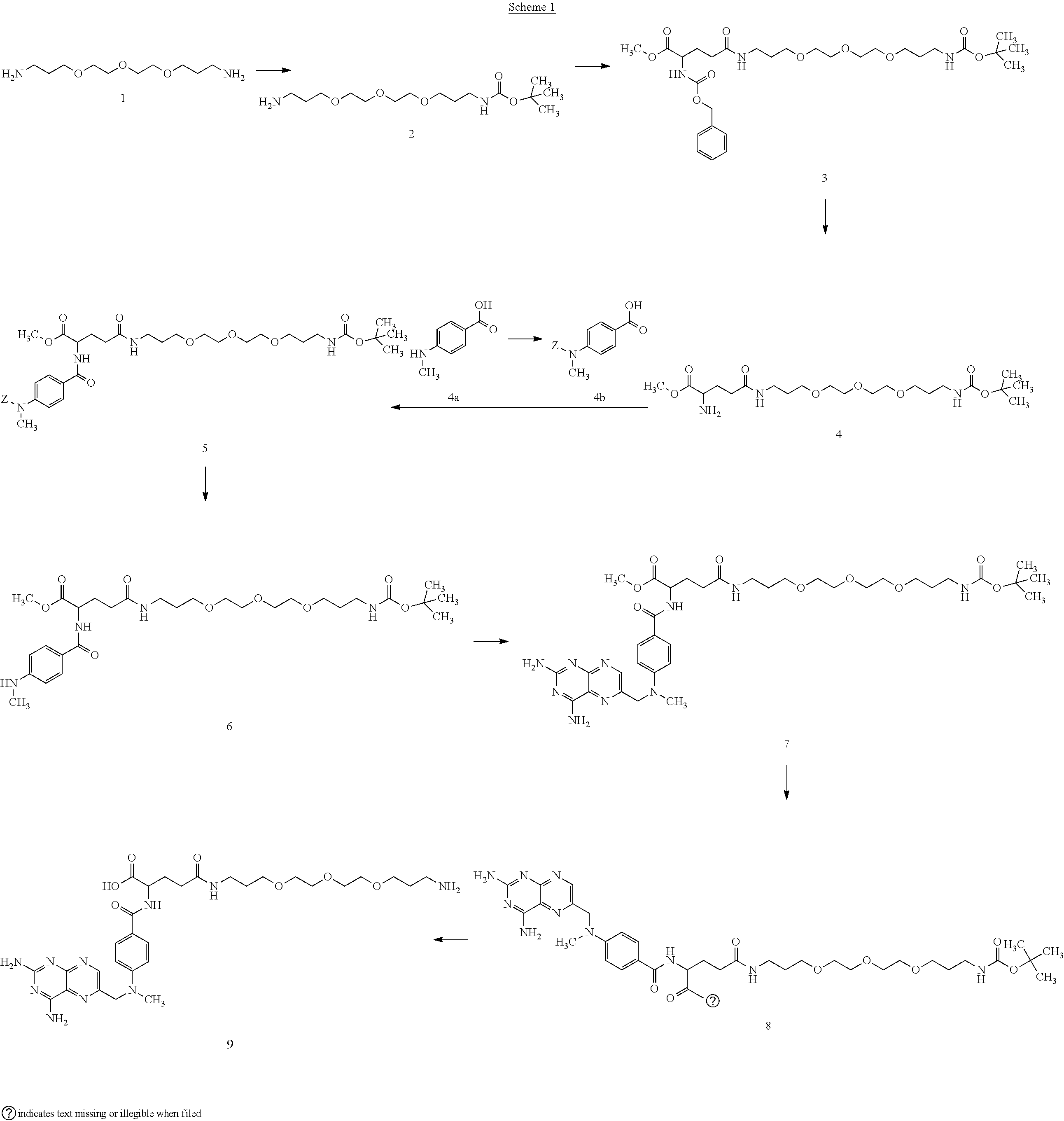

### Description of Synthetic Procedures

### 1.1. Synthesis of 4[4(3{2[2(3aminopropoxy)ethoxy]ethoxy}propyl)carbamoyl]2[(4{[(2,4-diaminopteridin-6-yl)methyl)](methyl)amino}phenyl) formamido]butanoic Acid

### Step: BOC-Protection

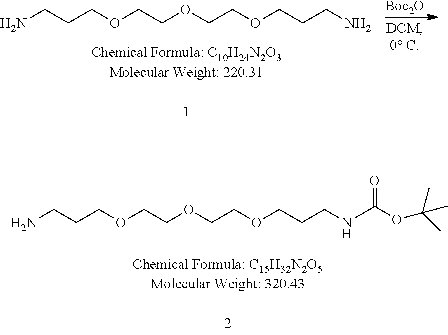 [SMILES: "Boc2O" ; NCCCOCCOCCOCCCN ; CC(C)(C)OC(=O)NCCCOCCOCCOCCCN]

### Reaction Conditions:

| Experiment performed | | | Results | | | | |
|---|---|---|---|---|---|---|---|
| No. | Type | Conditions | Amount | | Purity | Yield | Others |
| 1. | 1323-008 | 4,7,10-Trioxa-1,13-tridecanediamine | 0.48 | g | 95+% | 41.25% | Colorless |
| | | (2.0 g, 0.01 mol, 2.5Eq), Di-tert-butyl | | | | | oil |
| | | dicarbonate (0.79 g, 3.69 mmol, 1eq), | | | | | |
| | | DCM (60 mL, 30 vol.), 0° C. →25° C., | | | | | |
| | | 18 hrs (overnight). | | | | | |
| 2. | 1323-009 | 4,7,10-Trioxa-1,13-tridecanediamine | 41 | g | 95+% | 71.98% | Colorless |
| | | (98.0 g, 0.44 mol, 2.5Eq), Di-tert-butyl | | | | | oil |
| | | dicarbonate (38.83 g, 0.17 mol, 1eq), | | | | | |
| | | DCM (2.94 L, 30 vol.), 0° C. →25° C., | | | | | |
| | | 18 hrs (overnight). | | | | | |

### Apparatus

| No | Name |
|---|---|
| 1. | Three-neck round-bottom flask (4-L) equipped with magnetic |
| | stirrer bar, thermometer, bubbler and cooling bath |
| 2. | Cryostat |
| 3. | Rotavapor |
| 4. | Separatory funnel, filtering funnel |

### Raw Materials Consumption on 98 g Scale

| | | | g/batch | |
|---|---|---|---|---|
| Name | Mw | Equivalent | (ml/batch) | |
| 4,7,10-Trioxa-1,13-tridecanediamine | 220.31 | 1.0 | 28.8 | g |
| Di-tert-butyl dicarbonate | 218.25 | 0.4 | 16.72 | g |
| DCM | — | 10 vol. | 980.0 | ml |
| DCM | — | 20 vol. | 1960 | ml |

### Procedure

Bis-(3-aminopropyl)diethyleneglycol (98 g, 97.5 mL, 0.44 mol) was dissolved in DCM (1960 mL, 20 vol.) and Boc-anhydride (38.84 g, 0.177 mol) in DCM (980 mL, 10 vol.) was added dropwise within 4h at 0° C. The mixture was left to react overnight at room temperature. The reaction was completed according to TLC, the reaction mixture was evaporated to ˜0.5 L, the residue was washed four times with saturated NaCl solution (500 mL each) to remove excess diamine. The organic layer was quenched with 10% w. KHSO₄ to pH˜4. The organic phase was separated to remove doubly protected diamine and the aqueous phase was basified with 6N NaOH to pH˜10, extracted with DCM (3*250 mL), organic layer was successively washed with water and brine. The organic phase was dried (MgSO₄) and concentrated in vacuo to yield the title compound (41 g, 71.9 percent yield) as a colorless oil. TLC control (SiO₂, CHCl₃:MeOH:25% w NH₃ in H₂O=8:2:0.2):R_{f}=0.6, ninhydrin stain. ¹H NMR confirmed the structure & purity.

¹H NMR (400 MHz, Chloroform-d) δ 5.12 (s, 1H), 3.68-3.49 (m, 12H), 3.23 (q, J=6.3 Hz, 2H), 2.82 (t, J=6.7 Hz, 2H), 1.76 (q, J=6.6 Hz, 4H), 1.45 (s, 9H).

### Step: Amide Coupling:

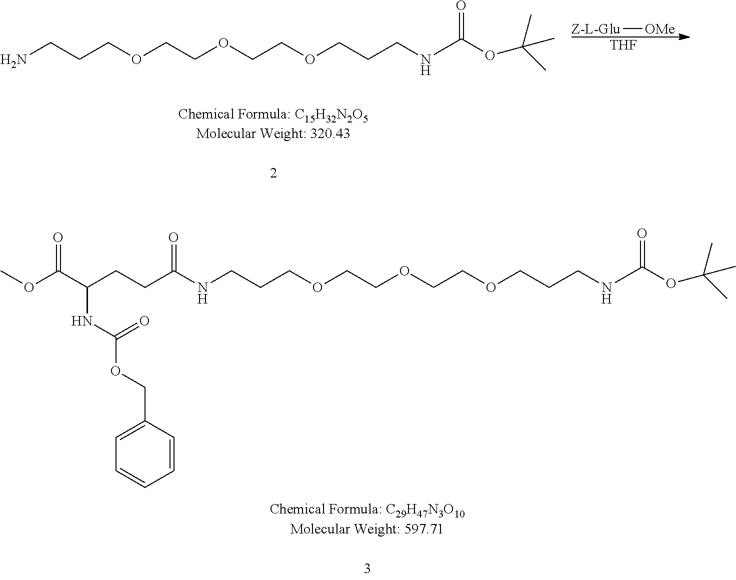

### Reaction Conditions:

| Experiment performed | | | Results | | | | |
|---|---|---|---|---|---|---|---|
| No. | Type | Conditions | Amount | | Purity | Yield | Others |
| 1. | 1323-010 | Z-L-Glu-OMe (0.38 g, 1.3 mmol, 1Eq), | 0.7 | g | 95+% By | 93% | Colorless |
| | | CDI (0.229 g, 1.4 mmol, 1.1Eq), tert- | | | ¹HNMR | | oil |
| | | butyl N-(3-{2-[2-(3-aminopropoxy)eth- | | | | | |
| | | oxy]ethoxy}propyl)carbamate | | | | | |
| | | (batch 1323-008) (0.47 g, 1.5 mmol, | | | | | |
| | | 1.15Eq) in THF (5.7 mL, 15 vol.), | | | | | |
| | | 25° C., 18 hrs (overnight). | | | | | |
| 2. | 1323-013 | Z-L-Glu-OMe (32 g, 0.108 mol, 1Eq), | 60 | g | 95+% By | 92.8% | yellowish |
| | | CDI (19.32 g, 0.119 mol, 1.1Eq), tert- | | | ¹HNMR | | oil |
| | | butyl N-(3-{2-[2-(3-aminopropoxy)eth- | | | | | |
| | | oxy]ethoxy}propyl)carbamate | | | | | |
| | | (batch 1323-010) (39.99 g, 0.125 | | | | | |
| | | mol, 1.15Eq) in THF (480 mL, 15 | | | | | |
| | | vol.), 25° C., 18 hrs (overnight). | | | | | |

### Apparatus

| No | Name |
|---|---|
| 1. | Three-neck round-bottom flask (1-L) equipped with |
| | magnetic stirrer bar thermometer and bubbler |
| 2. | Rotavapor. |
| 3. | Separating and Filtering funnels. |

Raw materials consumption on 32 g scale

| | | | g/batch | |
|---|---|---|---|---|
| Name | Mw | Equivalent | (ml/batch) | |
| Z-L-Glu-OMe | 295.3 | 1.0 | 32.0 | g |
| CDI | 162.1 | 1.1 | 19.32 | g |
| tert-butyl N-(3-{2-[2-(3- | 320.43 | 1.15 | 39.93 | g |
| aminopropoxy)ethoxy]eth- | | | | |
| oxy}propyl)carbamate | | | | |
| THF | — | 15 vol. | 480 | ml |

### Procedure

Z-L-Glu-OMe (32 g, 0.108 mol) was dissolved in THF (480 mL, 15 vol.), 1,1′-Carbonyldiimidazole (19.32 g, 0.119 mol) was added and the mixture was stirred for 45 minutes. tert-Butyl N-(3-{2-[2-(3-aminopropoxy)ethoxy]ethoxy}propyl)carbamate 1323-009 (39.93 g, 0.125 mol) was further added, and the mixture was stirred at 25° C. for 65 hrs. After completion of the reaction, THF was evaporated in vacuo, water (200 mL) was added to the residue, followed by extraction with ethyl acetate (150*3 mL). The organic layer was washed with 10% KHSO4, 1N NaOH, brine and then dried over anhydrous MgSO₄. The solvent was evaporated under reduced pressure to give 60.1 g (92.79% yield) of methyl 2-{[(benzyloxy)carbonyl]amino}4[(3{2[2(3{[(tertbutoxy)carbonyl]amino}propoxy)ethoxy]ethoxy}propyl)carbamoyl]butanoate as a yellowish oil.

### Note:

Compound was used in further steps without additional purification. ¹H NMR confirmed the structure & purity.

¹H NMR (400 MHz, Chloroform-d) δ 7.43-7.30 (m, 5H), 6.49 (s, 1H), 5.94 (s, 1H), 5.12 (s, 2H), 4.98 (s, 1H), 4.35 (td, J=8.3, 4.1 Hz, 1H), 3.75 (s, 3H), 3.59 (dddd, J=22.1, 16.4, 8.8, 4.3 Hz, 12H), 3.36 (qd, J=6.1, 1.9 Hz, 2H), 3.22 (q, J=6.3 Hz, 2H), 2.31-2.15 (m, 3H), 2.08-1.96 (m, 1H), 1.77 (p, J=6.1 Hz, 4H), 1.45 (s, 9H).

### Step: Z-Deprotection

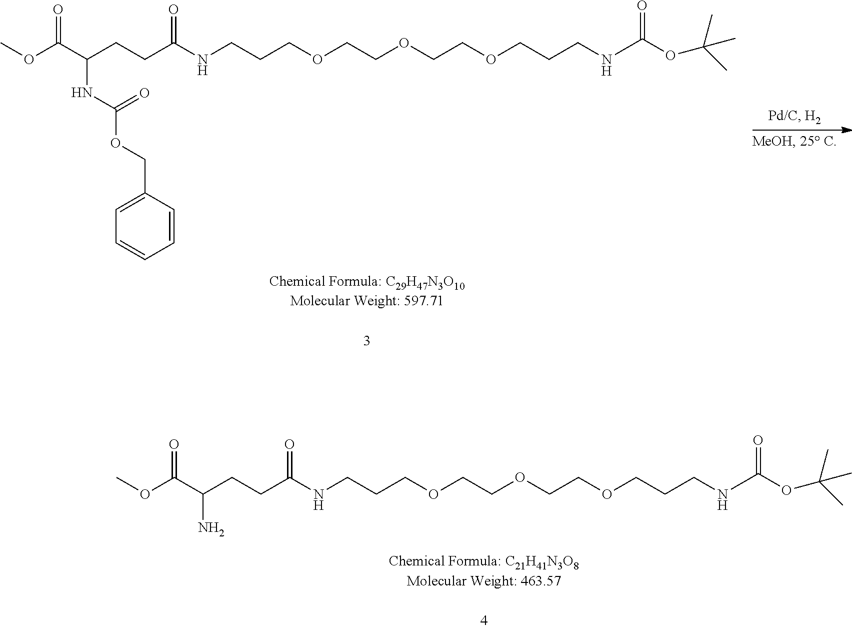

### Reaction Conditions:

| Experiment performed | | | Results | | | | |
|---|---|---|---|---|---|---|---|
| No. | Type | Conditions | Amount | | Purity | Yield | Others |
| 1. | 1323-012 | methyl 2-{[(benzyloxy)carbonyl]amino}- | 0.49 | g | 95+% | 97.2% | greenish |
| | | 4-[(3{2[2(3{[(tertbutoxy)carbonyl]ami- | | | | | oil |
| | | no}propoxy)ethoxy]ethoxy}propyl)car- | | | | | |
| | | bamoyl]butanoate 1323-010 (0.65 g, | | | | | |
| | | 1.09 mmol, 1Eq), 10% w Pd/C (0.065 g, | | | | | |
| | | 10 w %), in MeOH (32 mL, 50 vol.), | | | | | |
| | | hydrogen, ~15 psi, 25° C., 18 hrs | | | | | |
| | | (overnight). | | | | | |
| 2. | 1323-016 | methyl 2-{[(benzyloxy)carbonyl]amino}- | 45 | g | 95+% | 100% | greenish |
| | | 4-[(3-{2[2(3{[(tertbutoxy)carbonyl]ami- | | | | | oil |
| | | no}propoxy)ethoxy]ethoxy}propyl)car- | | | | | |
| | | bamoyl]butanoate 1323-013 (58 g, 0.097 | | | | | |
| | | mol, 1Eq), 10% w Pd/C (5.8 g, 10 w %), in | | | | | |
| | | MeOH (290 mL, 5 vol.), hydrogen, ~45 | | | | | |
| | | psi, 25° C., 18 hrs (overnight). | | | | | |

### Apparatus

| No | Name |
|---|---|
| 1. | Parr shaker type hydrogenation apparatus |
| 2. | Rotavapor. |
| 3. | Filtering funnel. |

### Raw Materials Consumption on 58 g Scale

| | | | g/batch | |
|---|---|---|---|---|
| Name | Mw | Equivalent | (ml/batch) | |
| methyl 2-{[(benzyloxy)carbonyl]ami- | 597.71 | 1.00 | 58 | g |
| no}-4-[(3{2[2(3{[(tertbutoxy)carbon- | | | | |
| yl]amino}propoxy)ethoxy]ethoxy}pro- | | | | |
| pyl)carbamoyl]butanoate | | | | |
| Palladium on activated carbon | — | 0.01 | 5.8 | g |
| MeOH | — | 5 vol. | 290 | mL |

### Procedure

To the solution of methyl 2-{[(benzyloxy)carbonyl]amino}-4[(3{2[2 (3{[(tertbutoxy)carbonyl]amino}propoxy)ethoxy]ethoxy}propyl) carbamoyl]butanoate 1323-013 (58 g, 0.097 mol) in MeOH (290 mL, 5 vol.) was added 5.8 g (10w %) of Palladium 10% w on activated carbon. Reaction was hydrogenated in the Parr shaker type hydrogenation apparatus at ˜3.5 atm, 25° C., 18h. After reaction was completed, the reaction mixture was filtered through pad of celite and concentrated in vacuo. Yield crude: 45 g (yield: quantitative) as a greenish oil.

### Note:

The crude product was used in the next step. TLC control (SiO₂, CH₂Cl₂:MeOH=9:1):R_{f}=0.4, ninhydrin stain. ¹H NMR confirmed the structure & purity.

¹H NMR (400 MHz, Chloroform-d) δ 6.46 (s, 1H), 5.04 (s, 1H), 3.73 (s, 3H), 3.67-3.48 (m, 12H), 3.37 (q, J=6.1 Hz, 2H), 3.22 (q, J=6.4 Hz, 2H), 2.36-2.24 (m, 2H), 1.96 (s, 3H), 1.88-1.64 (m, 5H), 1.44 (s, 9H).

### Step: Amide Coupling (Z-Protection)

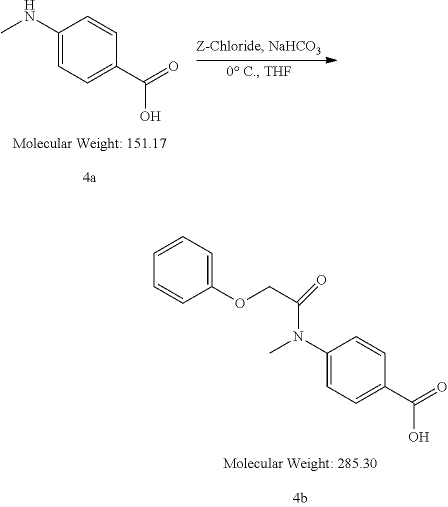 [SMILES: CNc1ccc(C(=O)O)cc1 ; CN(C(=O)COc1ccccc1)c1ccc(C(=O)O)cc1]

### Reaction Conditions:

| Experiments performed | | | Results | | | | |
|---|---|---|---|---|---|---|---|
| No. | Type | Conditions | Amount | | Purity | Yield | Others |
| 1. | 1323-011 | 4-(Methylamino)benzoic acid | 0.9 | g | 100% | 47.68% | Solid |
| | | (1 g, 6.62 mmol, 1Eq) Benzyl | | | | | white |
| | | chloroformate (1.13 mL, 7.94 | | | | | |
| | | mmol, 1.2Eq), Sodium | | | | | |
| | | bicarbonate (1.334 g, 15.88 | | | | | |
| | | mmol, 2.4Eq) in THF (10 mL, | | | | | |
| | | 10 vol.), 0° C.→25° C., 4 hrs. | | | | | |
| 2. | 1323-014 | 4-(Methylamino)benzoic acid | 25.8 | g | 100% | 91.13% | Solid |
| | | (15 g, 0.099 mol, 1Eq) Benzyl | | | | | white |
| | | chloroformate (16.99 mL, 0.119 | | | | | |
| | | mol, 1.2Eq), Sodium bicarbonate | | | | | |
| | | (20.08 g, 0.238 mol, 2.4Eq) | | | | | |
| | | in THF (150 mL, 10 vol.), | | | | | |
| | | 0° C.→25° C., 18 hrs | | | | | |
| | | (overnight). | | | | | |

### Apparatus

| No | Name |
|---|---|
| 1. | RBF (0.25 L) equipped with magnetic stirrer bar, |
| | thermometer, cooling bath |
| 2. | Dropping funnel (25 ml), Separatory funnel (0.25 L) |
| 3. | Rotavapor, Filter flask, Büchner filter funnel |

### Raw Materials Consumption on 15 g Scale

| | | | g/batch | |
|---|---|---|---|---|
| Name | Mw | Equivalent | (ml/batch) | |
| 4-(Methylamino)benzoic acid | 151.16 | 1.0 | 15 | g |
| Benzyl chloroformate | 170.59 | 1.2 | 20.3 g/16.99 mLg | |
| Sodium bicarbonate | 84.01 | 2.4 | 20.008 | g |
| THF | — | 10.0 vol. | 150.0 | ml |

### Procedure

To a stirred solution of 4-(methylamino)benzoic acid (15 g, 0.099 mol) in dry THF (150 mL, 10 vol.) at 0° C. was added NaHCO₃ (20.08 g, 0.238 mol) and benzyl chloroformate (16.99 mL, 0.119 mol), the reaction mixture was allowed to warm to room temperature and the mixture was stirred for 18h. The reaction mixture was filtered. The filtrate was concentrated under reduced pressure, and the residue was dissolved in 1 M aqueous NaOH. The aqueous solution was washed with Et₂O, acidified with 6 M hydrochloric acid to pH 3, and extracted with AcOEt. The organic layer was dried over anhydrous MgSO₄ and concentrated in vacuo to afford (Benzyloxycarbonyl)-4-(methylamino)benzoic acid as colorless particles (25.8 g, 91.13% yield).

¹H NMR confirmed the structure & purity

¹H NMR (400 MHz, Chloroform-d) δ 8.18-8.05 (m, 2H), 7.46-7.40 (m, 2H), 7.40-7.30 (m, 5H), 5.24 (s, 2H), 3.41 (s, 3H).

NMR

### Step: Amide Coupling

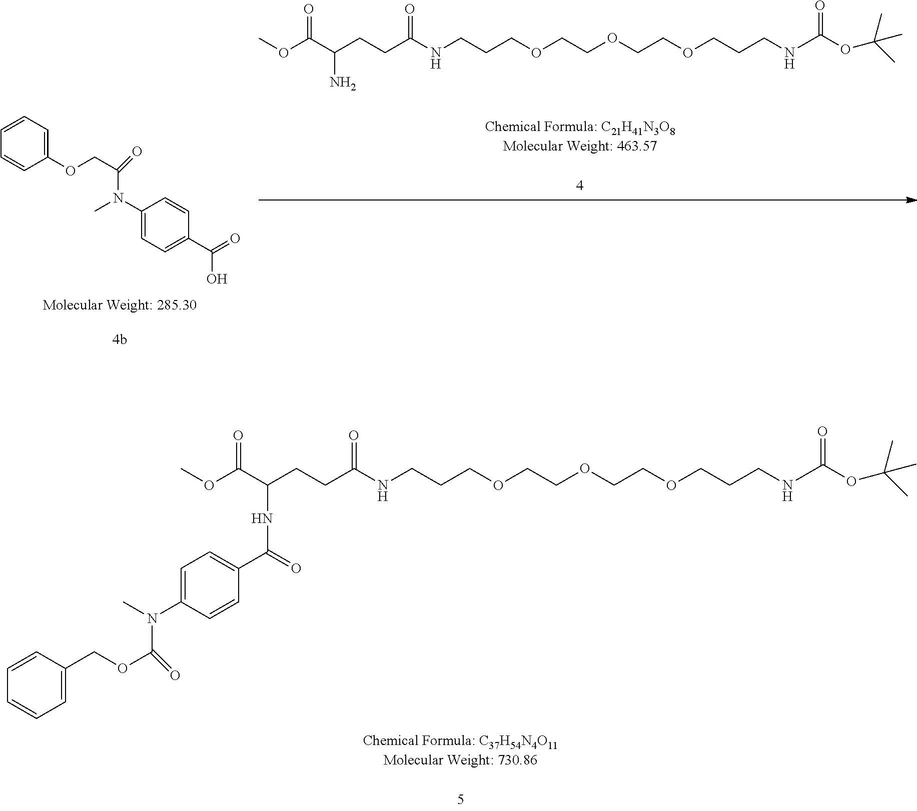 [SMILES: COC(=O)C(N)CCC(=O)NCCCOCCOCCOCCCNC(=O)OC(C)(C)C ; CN(C(=O)COc1ccccc1)c1ccc(C(=O)O)cc1 ; COC(=O)C(CCC(=O)NCCCOCCOCCOCCCNC(=O)OC(C)(C)C)NC(=O)c1ccc(N(C)C(=O)OCc2ccccc2)cc1]

### Reaction Conditions:

| Experiment performed | | | Results | | | | |
|---|---|---|---|---|---|---|---|
| No. | Type | Conditions | Amount | | Purity | Yield | Others |
| 1. | 1323-015 | 4-{[(benzyloxy)carbonyl](meth- | 0.21 | g | 96% | 31% | Oil, |
| | | yl)amino}benzoic acid 1323-011 | | | | | colorless |
| | | (0.26 g, 0.9 mmol, 1Eq), CDI | | | | | |
| | | (0.163 g, 1.0 mmol, 1.1Eq), | | | | | |
| | | methyl 2-amino-4-[(3-{2-[2-(3- | | | | | |
| | | {[(tert-butoxy)carbonyl]ami- | | | | | |
| | | no}propoxy)ethoxy]ethoxy}pro- | | | | | |
| | | pyl)carbamoyl] butanoate batch | | | | | |
| | | 1323-012 (0.48 g, 1.1 mmol, | | | | | |
| | | 1.15Eq) in THF (3.9 mL, 15 | | | | | |
| | | vol.), 25° C., 18 hrs (overnight). | | | | | |
| 2. | 1323-017 | 4-{[(benzyloxy)carbonyl](meth- | 1.5 | g | 94.7% | 95.14% | Oil, |
| | | yl)amino}benzoic acid 1323-014 | | | | | colorless |
| | | (0.615 g, 2.16 mmol), EDC*HCl | | | | | |
| | | (0.455 g, 2.37 mmol), HOBT (0.321 | | | | | |
| | | g, 2.37 mmol), methyl 2-amino-4- | | | | | |
| | | [(3-{2-[2-(3-{[(tertbutoxy)car- | | | | | |
| | | bonyl]amino}propoxy)ethoxy]eth- | | | | | |
| | | oxy}propyl)carbamoyl]butanoate | | | | | |
| | | batch 1323-016 (1 g, 2.16 mmol), | | | | | |
| | | DIPEA (1.5 mL, 8.63 mmol), ACN | | | | | |
| | | (20 mL, 20 vol), | | | | | |
| | | 24° C. →0° C.→24° C.; 24 h. | | | | | |
| 3 | 1323-019 | 4-{[(benzyloxy)carbonyl](meth- | 49 | g | 95.9% | 88.8% | Oil, |
| | | yl)amino}benzoic acid 1323-014 | | | | | colorless |
| | | (21.54 g, 0.075 mol), EDC*HCl | | | | | |
| | | (15.92 g, 0.083 mol), HOBT | | | | | |
| | | (11.22 g, 0.083 mol), methyl | | | | | |
| | | 2-amino-4-[(3-{2-[2-(3- | | | | | |
| | | {[(tertbutoxy)carbonyl]ami- | | | | | |
| | | no}propoxy)ethoxy]ethoxy}pro- | | | | | |
| | | pyl)carbamoyl]butanoate batch | | | | | |
| | | 1323-016 (35 g, 0.075 mol), | | | | | |
| | | DIPEA (52.74 mL, 0.3 mol), | | | | | |
| | | ACN (525 mL, 15 vol), | | | | | |
| | | 24° C. →0° C.→24° C.; 24 h | | | | | |

### Apparatus

| No | Name |
|---|---|
| 1. | RBF (1 L) equipped with magnetic stirrer bar, |
| | thermometer, cooling bath |
| 2. | Dropping funnel (50 ml), Separatory funnel (0.5 L) |
| 3. | Rotavapor, Filter flask, Büchner filter funnel |

Raw materials consumption on 35 g scale

| | | | g/batch | |
|---|---|---|---|---|
| Name | Mw | Equivalent | (ml/batch) | |
| methyl 2-amino-4-[(3-{2-[2-(3- | 187.67 | 1.0 | 35 | g |
| {[(tertbutoxy)carbonyl]ami- | | | | |
| no}propoxy)ethoxy]ethoxy}pro- | | | | |
| pyl)carbamoyl]butanoate | | | | |
| 4{[(benzyloxy)carbonyl](meth- | 228.24 | 1.0 | 21.54 | g |
| yl)amino}benzoic acid | | | | |
| HOBT | 135.12 | 1.1 | 11.22 | g |
| EDC HCl | 191.17 | 1.1 | 15.92 | g |
| DIPEA | 129.24 | 4.0 | 39.03 g/52.74 mL | |
| ACN | — | 15.0 vol. | 525.0 | ml |

### Procedure

4-{[(benzyloxy)carbonyl](methyl)amino}benzoic acid 1323-014 21.54 g (0.075 mol, 1 eq), 1-Hydroxybenzotriazole 11.222 g (0.083 mol, 1.1 eq), N-(3-Dimethylaminopropyl)-N′-ethylcarbodiimide hydrochloride 15.92 g (0.083 mol, 1.1 eq) were dissolved in CH₃CN (525 ml, 15 vol.). The resulting solution was agitated at 20° C. for 1h and charged with 35 g (0.075 mol, 1 eq) of methyl 2-amino-4-[(3-{2-[2-(3-{[(tertbutoxy)carbonyl]amino}propoxy)ethoxy]ethoxy}propyl)carbamoyl]butanoate 1323-016. The RM was cooled to about 0° C. and 52.74 ml (0.3 mol, 4 eq) N,N-Diisopropylethylamine was added over 25 minutes while maintaining a temperature below 10° C. The solution was slowly heated to 24° C. and held at 20° C. for 65 hours (over weekend). After completion of the reaction, ACN was evaporated in vacuo, water (250 mL) was added to the residue, followed by extraction with ethyl acetate (150×3 mL). The organic layer was washed with 10% KHSO₄, 1N NaOH, brine and then dried over anhydrous MgSO₄. The solvent was evaporated under reduced pressure to give 49 g (88.8% yield) of title product as a colorless oil.

¹H NMR (400 MHz, Chloroform-d) δ 8.10 (s, 1H), 7.95-7.84 (m, 2H), 7.44-7.30 (m, 7H), 6.70 (s, 1H), 5.20 (s, 2H), 4.99 (s, 1H), 4.67 (ddd, J=8.0, 6.6, 4.6 Hz, 1H), 3.77 (s, 3H), 3.67-3.48 (m, 12H), 3.44-3.29 (m, 5H), 3.21 (q, J=6.3 Hz, 2H), 2.49-2.15 (m, 4H), 1.80-1.66 (m, 4H), 1.45 (s, 9H).

### Step: Z-Deprotection #2

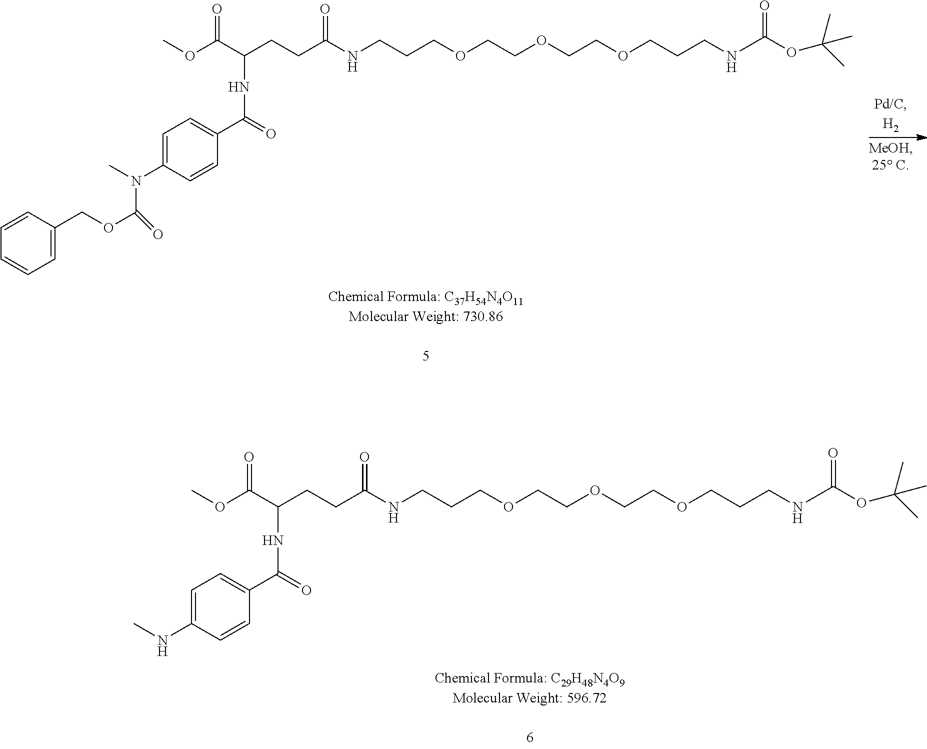

### Reaction Conditions:

| Experiment performed | | | Results | | | | |
|---|---|---|---|---|---|---|---|
| No. | Type | Conditions | Amount | | Purity | Yield | Others |
| 1. | 1323-018 | methyl2[(4{[(benzyloxy)carbon- | 1.2 | g | 90.4% | quantitative | Colorless |
| | | yl](methyl)amino}phenyl)form- | | | | | oil |
| | | amido]4[3{2[2(3{[(tertbuto- | | | | | |
| | | xy)carbonyl]amino}propoxy)eth- | | | | | |
| | | oxy]ethoxy}propyl)carba- | | | | | |
| | | moyl]butanoate 1323-017 (1.45 | | | | | |
| | | g, 1.98 mmol, 1Eq), 10% w | | | | | |
| | | Pd/C (0.15 g, 10 w %), in | | | | | |
| | | MeOH (29 mL, 20 vol.), | | | | | |
| | | hydrogen, ~15 psi, 25° C., | | | | | |
| | | 18 hrs (overnight). | | | | | |
| 2. | 1323-022 | methyl2[(4{[(benzyloxy)carbon- | 38.5 | g | 91.2% | 97.23% | Colorless |
| | | yl](methyl)amino}phenyl)form- | | | | | oil |
| | | amido]4[3{2[2(3{[(tertbuto- | | | | | |
| | | xy)carbonyl]amino}propoxy)eth- | | | | | |
| | | oxy]ethoxy}propyl)carba- | | | | | |
| | | moyl]butanoate 1323-019 (49 g, | | | | | |
| | | 0.066 mol, 1Eq), 10% w Pd/C | | | | | |
| | | (4.9 g, 10 w %) , in MeOH (340 | | | | | |
| | | mL, 7 vol.), hydrogen, ~45 psi, | | | | | |
| | | 25° C., | | | | | |

### Apparatus

| No | Name |
|---|---|
| 1. | Parr shaker type hydrogenation apparatus |
| 2. | Rotavapor. |
| 3. | Filtering funnel. |

Raw materials consumption on 48.5 g scale

| | | | g/batch | |
|---|---|---|---|---|
| Name | Mw | Equivalent | (ml/batch) | |
| methyl2[(4{[(benzyloxy)carbon- | 730.86 | 1.00 | 48.5 | g |
| yl](methyl)amino}phenyl)form- | | | | |
| amido]4[3{2[2(3{[(tertbutoxy)car- | | | | |
| bonyl]amino}propoxy)ethoxy]eth- | | | | |
| oxy}propyl)carbamoyl]butanoate | | | | |
| Palladium on activated carbon | — | 0.01 | 4.85 | g |
| MeOH | — | 7 vol. | 339 | mL |

### Procedure

To the solution of methyl2[(4{[(benzyloxy)carbonyl](methyl)amino}phenyl)formamido]4[3{2 [2(3{[(tertbutoxy)carbonyl]amino}propoxy)ethoxy]ethoxy}propyl)carbamoyl]butanoate 1323-019 (48.5 g, 0.066 mol) in MeOH (340 mL, 7 vol.) was added 4.9 g (10w %) of Palladium 10% w on activated carbon.

Reaction was hydrogenated in the Parr shaker type hydrogenation apparatus at ˜3.5 atm, 25° C., 18h.

After reaction was completed, the reaction mixture was filtered through pad of celite and concentrated in vacuo.

Yield: 38.5 g (yield: 97.23%) as a yellowish oil.

¹H NMR confirmed the structure & purity.

¹H NMR (400 MHz, Chloroform-d) δ 7.83-7.63 (m, 2H), 7.39 (d, J=6.9 Hz, 1H), 6.73 (s, 1H), 6.62-6.52 (m, 2H), 5.03 (s, 1H), 4.69 (ddd, J=8.8, 7.1, 4.1 Hz, 1H), 4.20 (s, 1H), 3.76 (s, 3H), 3.68-3.47 (m, 12H), 3.34 (tt, J=13.7, 6.7 Hz, 2H), 3.21 (q, J=6.3 Hz, 2H), 2.88 (s, 3H), 2.45-2.10 (m, 4H), 1.75 (dd, J=9.0, 3.7 Hz, 4H), 1.44 (s, 9H).

### Step: Alkylation

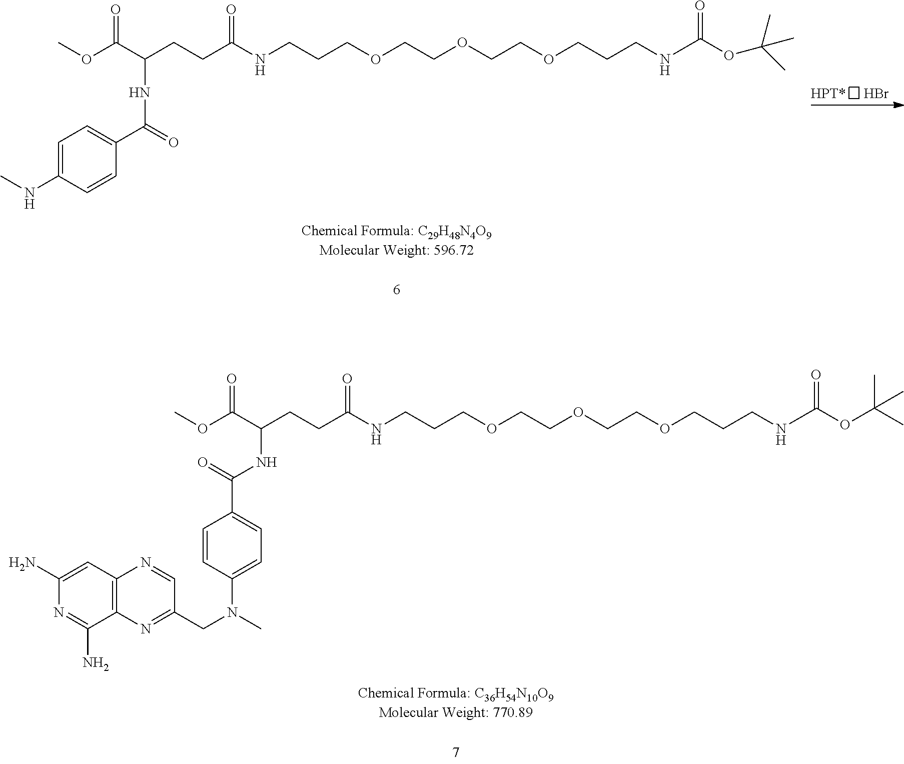 [SMILES: CNc1ccc(C(=O)NC(CCC(=O)NCCCOCCOCCOCCCNC(=O)OC(C)(C)C)C(=O)OC)cc1 ; COC(=O)C(CCC(=O)NCCCOCCOCCOCCCNC(=O)OC(C)(C)C)NC(=O)c1ccc(N(C)Cc2cnc3cc(N)nc(N)c3n2)cc1]

### Reaction Conditions:

| Experiment performed | | | Results | | | | |
|---|---|---|---|---|---|---|---|
| No. | Type | Conditions | Amount | | Purity | Yield | Others |
| 1. | 1323-020 | methyl4[(3{2[2(3{[(tertbu- | n/a | | 43.8% of | n/a | n/a |
| | | toxy)carbonyl]amino}pro- | | | SM and | | |
| | | poxy)ethoxy]ethoxy}pro- | | | 23.7% of | | |
| | | pyl)carmoyl]-2-{[4-(methyl- | | | DP after | | |
| | | amino)phenyl]formamido}bu- | | | 2.5 h | | |
| | | tanoate1323-018 (0.175 g, | | | 17.2% of | | |
| | | 0.29 mmol, 1Eq), 2,4-di- | | | SM and | | |
| | | aminopteridine hydrobromide | | | 21.5% of | | |
| | | (0.128 g, 0.4 mmol, 1.4Eq.), | | | DP after | | |
| | | sodium bicarbonate (0.074 g, | | | 2.5 d | | |
| | | 0.88 mmol, 3Eq.), in CH₃CN | | | | | |
| | | (3.5 mL, 20 vol.), 65° C., 50 h. | | | | | |
| 2. | 1323-021 | methyl4[(3{2[2(3{[(tertbu- | 0.18 | g | n/a | n/a | orange |
| | | toxy)carbonyl]amino}pro- | | | | | oil |
| | | poxy)ethoxy]ethoxy}pro- | | | | | |
| | | pyl)carmoyl]-2-{[4-(methyl- | | | | | |
| | | amino)phenyl]formamido}bu- | | | | | |
| | | tanoate1323-018 (0.175 g, | | | | | |
| | | 0.29 mmol, 1Eq), 2,4-di- | | | | | |
| | | aminopteridine hydrobromide | | | | | |
| | | (0.128 g, 0.4 mmol, 1.4Eq.) | | | | | |
| | | in DMA (1.8 mL, 10 vol.), | | | | | |
| | | 65° C., 18 h (overnight) | | | | | |
| 3 | 1323-023 | methyl4[(3{2[2(3{[(tertbu- | n/a | | 13.9% of | n/a | n/a |
| | | toxy)carbonyl]amino}pro- | | | SM and | | |
| | | poxy)ethoxy]ethoxy}pro- | | | 5.9% of | | |
| | | pyl)carmoyl]-2-{[4-(methyl- | | | DP | | |
| | | amino)phenyl]formamido}bu- | | | | | |
| | | tanoate1323-018 (0.175 g, | | | | | |
| | | 0.29 mmol, 1Eq), 2,4-di- | | | | | |
| | | aminopteridine hydrobromide | | | | | |
| | | (0.128 g, 0.4 mmol, 1.4Eq.), | | | | | |
| | | DIPEA (0.255 mL, 1.47 mmol, | | | | | |
| | | 5Eq.), in DMA (1.8 mL, | | | | | |
| | | 10 vol.), 65° C., 7 h. | | | | | |
| 4 | 1323-024 | methyl4[(3{2[2(3{[(tertbu- | n/a | | 14.2% of | n/a | n/a |
| | | toxy)carbonyl]amino}pro- | | | SM and | | |
| | | poxy)ethoxy]ethoxy}pro- | | | 10.6% of | | |
| | | pyl)carmoyl]-2-{[4-(methyl- | | | DP | | |
| | | amino)phenyl]formamido}bu- | | | | | |
| | | tanoate1323-018 (0.175 g, | | | | | |
| | | 0.29 mmol, 1Eq), 2,4-di- | | | | | |
| | | aminopteridine hydrobromide | | | | | |
| | | (0.128 g, 0.4 mmol, 1.4Eq.), | | | | | |
| | | sodium bicarbonate (0.197 | | | | | |
| | | mL, 2.35 mmol, 8Eq.), in | | | | | |
| | | DMA (1.8 mL, 10 vol.), | | | | | |
| | | 65° C., 18 h | | | | | |
| 5 | 1323-025 | methyl4[(3{2[2(3{[(tertbu- | 0.13 | g | 90.2% | 17.04% | Yellow |
| | | toxy)carbonyl]amino}pro- | | | | | oil |
| | | poxy)ethoxy]ethoxy}pro- | | | | | |
| | | pyl)carmoyl]-2-{[4-(methyl- | | | | | |
| | | amino)phenyl]formamido}bu- | | | | | |
| | | tanoate1323-022 (0.59 g, | | | | | |
| | | 0.99 mmol, 1Eq), 2,4-di- | | | | | |
| | | aminopteridine hydrobromide | | | | | |
| | | (0.499 g, 1.48 mmol, 1.5Eq.) | | | | | |
| | | in DMA (1.8 mL, 10 vol.), | | | | | |
| | | 65° C., 4.5 h | | | | | |
| 6 | 1323-027 | methyl4[(3{2[2(3{[(tertbu- | 1.2 | g | 81.5% | 19.83% | Yellow |
| | | toxy)carbonyl]amino}pro- | | | | | oil |
| | | poxy)ethoxy]ethoxy}pro- | | | | | |
| | | pyl)carmoyl]-2-{[4-(methyl- | | | | | |
| | | amino)phenyl]formamido}bu- | | | | | |
| | | tanoate1323-022 (4.68 g, | | | | | |
| | | 7.85 mmol, 1Eq), 2,4-di- | | | | | |
| | | aminopteridine hydrobromide | | | | | |
| | | (3.95 g, 11.77 mmol, 1.5Eq.) | | | | | |
| | | in DMA (46.8 mL, 10 vol.), | | | | | |
| | | 65° C., 4.5 h | | | | | |
| 7 | 1323-028 | methyl4[(3{2[2(3{[(tertbu- | 3.2 | g | 98.9% | 41.95% | Yellow |
| | | toxy)carbonyl]amino}pro- | | | | | foam |
| | | poxy)ethoxy]ethoxy}pro- | | | | | |
| | | pyl)carmoyl]-2-{[4-(methyl- | | | | | |
| | | amino)phenyl]formamido}bu- | | | | | |
| | | tanoate1323-022 (5.9 g, | | | | | |
| | | 9.9 mmol, 1Eq), 2,4-di- | | | | | |
| | | aminopteridine hydrobromide | | | | | |
| | | (4.99 g, 14.8 mmol, 1.5Eq.) | | | | | |
| | | in DMA (59 mL, 10 vol.), | | | | | |
| | | 55° C., 3 h | | | | | |
| 8 | 1323-029 | methyl4[(3{2[2(3{[(tertbu- | 7.5 | g | 98.9% | 36.25% | Yellow |
| | | toxy)carbonyl]amino}pro- | | | | | foam |
| | | poxy)ethoxy]ethoxy}pro- | | | | | |
| | | pyl)carmoyl]-2-{[4-(methyl- | | | | | |
| | | amino)phenyl]formamido}bu- | | | | | |
| | | tanoate1323-022 (16 g, | | | | | |
| | | 26.84 mmol, 1Eq), 2,4-di- | | | | | |
| | | aminopteridine hydrobromide | | | | | |
| | | (13.52 g, 40.25 mmol, 1.5Eq.) | | | | | |
| | | in DMA (160 mL, 10 vol.), | | | | | |
| | | 55° C., 3 h | | | | | |

### Apparatus

| No | Name |
|---|---|
| 1. | RBF (0.25 L) equipped with magnetic stirrer bar, |
| | thermometer, heating bath |
| 2. | Separatory funnel (0.5 L) |
| 3. | Rotavapor, Filter flask, Büchner filter funnel |

Raw materials consumption on 16 g scale

| | | | g/batch | |
|---|---|---|---|---|
| Name | Mw | Equivalent | (ml/batch) | |
| methyl4[(3{2[2(3{[(tertbutoxy)car- | 596.2 | 1.00 | 16 | g |
| bonyl]amino}propoxy)ethoxy]eth- | | | | |
| oxy}propyl)carmoyl]-2-{[4-(methyl- | | | | |
| amino)phenyl]formamido}butanoate | | | | |
| 2,4-diaminopteridine Hydrobromide | 335.99 | 1.5 | 13.525 | g |
| DMA | — | 10 vol. | 160 | mL |

### Procedure

To a stirred solution of methyl4[(3{2[2 (3{[(tertbutoxy)carbonyl]amino}propoxy)ethoxy]ethoxy}propyl)carmoyl]-2-{1[4-(methylamino)phenyl]formamido}butanoate 1323-022 (16 g, 26.84 mmol, 1 Eq.) in dry DMA (160 mL, 10 vol.) at room temperature was added 2,4-diaminopteridine hydrobromide (13.52 g, 40.25 mmol, 1.5 Eq.), and the mixture was stirred at 55° C. for 3h. After reaction was completed, the reaction mixture was concentrated in vacuo, treated with aq. sat. NaHCO₃, extracted with EtOAc (several impurities were separated), then extracted with DCM (3×250 mL), the organic layers were combined, washed with water, brine and then dried over anhydrous MgSO₄. The solvent was evaporated under reduced pressure to give 17 g of crude methyl 4-[(3-{2-[2-(3-{[(tert-butoxy)carbonyl]amino}propoxy)ethoxy]ethoxy}propyl)carbamoyl]-2-[(4-{[(2,4-diaminopteridin-6-yl)methyl](methyl)amino}phenyl)formamido]butanoate as a yellow oil. Compound was purified using FC (EtOAc:MeOH—gradient elution). Obtained the title compound as a yellow foam. Yield: 7.5 g (36.25%). ¹H NMR confirmed the structure & purity

### Note:

The sample contains 28.57% mol or 4.38% w. of EtOAc

¹H NMR (400 MHz, DMSO-d6) δ 8.58 (s, 1H), 8.38 (d, J=7.2 Hz, 1H), 7.80 (t, J=5.6 Hz, 1H), 7.75-7.72 (m, 2H), 7.69 (s, 1H), 7.48 (s, 1H), 6.86-6.79 (m, 2H), 6.66 (s, 2H), 4.80 (s, 2H), 4.34 (q, J=7.5, 5.6 Hz, 1H), 3.62 (s, 3H), 3.54-3.40 (m, 8H), 3.37 (td, J=6.4, 2.6 Hz, 4H), 3.22 (s, 3H), 3.07 (q, J=6.5 Hz, 2H), 2.96 (q, J=6.6 Hz, 2H), 2.23-2.14 (m, 2H), 2.11-2.01 (m, 1H), 1.94 (d, J=9.0 Hz, 1H), 1.59 (p, J=6.7 Hz, 4H), 1.37 (s, 9H).

### Step: Hydrolysis of Ester

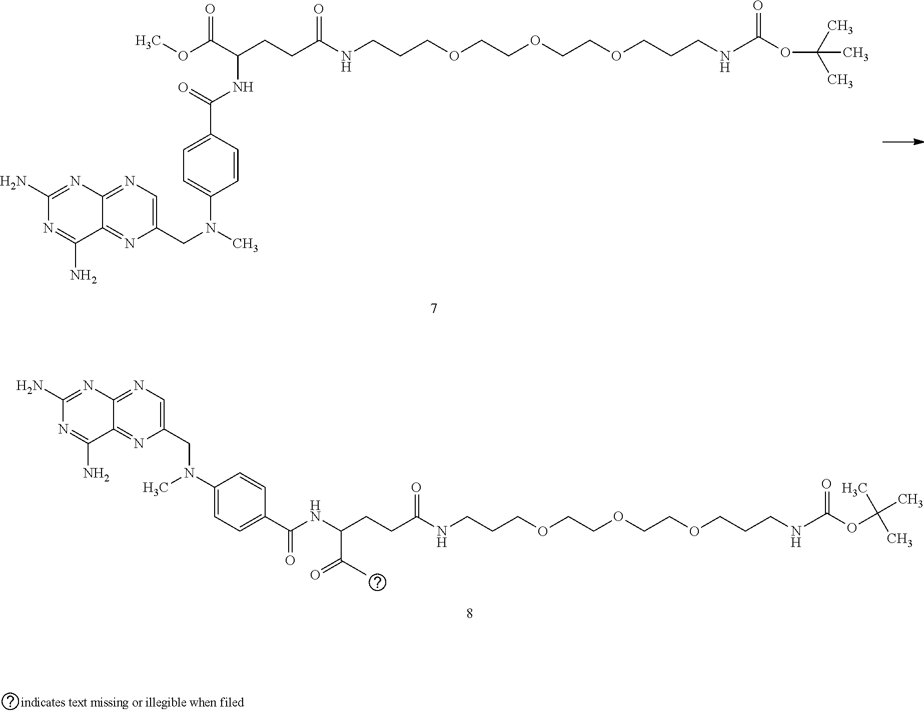 [SMILES: COC(=O)C(CCC(=O)NCCCOCCOCCOCCCNC(=O)OC(C)(C)C)NC(=O)c1ccc(N(C)Cc2cnc3nc(N)nc(N)c3n2)cc1 ; CC(=O)C(CCC(=O)NCCCOCCOCCOCCCNC(=O)OC(C)(C)C)NC(=O)c1ccc(N(C)Cc2cnc3nc(N)nc(N)c3n2)cc1]

### Reaction Conditions:

| Experiment performed | | | Results | | | | |
|---|---|---|---|---|---|---|---|
| No. | Type | Conditions | Amount | | Purity | Yield | Others |
| 1. | 1323-033 | methyl4[(3{2[2(3{[(tertbu- | 1.68 | g | 97.2% | Quantit. | orange |
| | | toxy)carbonyl]amino}pro- | | | | | solid |
| | | poxy)ethoxy]ethoxy}pro- | | | | | |
| | | pyl)carmoyl]-2-{[4-(methyl- | | | | | |
| | | amino)phenyl]formamido}bu- | | | | | |
| | | tanoate 1323-028 (1.7 g, | | | | | |
| | | 2.21 mmol, 1Eq), LiOH*H₂O | | | | | |
| | | (0.185 g, 4.42 mmol, 2Eq.), | | | | | |
| | | in MeOH (34 mL, 20 vol.), | | | | | |
| | | 25° C., 4 h. | | | | | |
| 2. | 1323-048 | methyl4[(3{2[2(3{[(tertbu- | 0.99 | g | n/a | Quantit | orange |
| | | toxy)carbonyl]amino}pro- | | | | | solid |
| | | poxy)ethoxy]ethoxy}pro- | | | | | |
| | | pyl)carmoyl]-2-{[4-(methyl- | | | | | |
| | | amino)phenyl]formamido}bu- | | | | | |
| | | tanoate 1323-029 (1 g, | | | | | |
| | | 1.3 mmol, 1Eq), LiOH*H₂O | | | | | |
| | | (0.109 g, 2.6 mmol, 2Eq.), | | | | | |
| | | in MeOH (20 mL, 20 vol.), | | | | | |
| | | 25° C., 4 h. | | | | | |
| 2. | 1323-049 | methyl4[(3{2[2(3{[(tertbu- | 4.7 | g | 93.6% | Quantit | orange |
| | | toxy)carbonyl]amino}pro- | | | | | solid |
| | | poxy)ethoxy]ethoxy}pro- | | | | | |
| | | pyl)carmoyl]-2-{[4-(methyl- | | | | | |
| | | amino)phenyl]formamido}bu- | | | | | |
| | | tanoate 1323-029 (4.75 g, | | | | | |
| | | 6.16 mmol, 1Eq), LiOH*H₂O | | | | | |
| | | (0.517 g, 12.32 mmol, 2Eq.), | | | | | |
| | | in MeOH (34 mL, 20 vol.), | | | | | |
| | | 25° C., 4 h. | | | | | |

### Apparatus

| No | Name |
|---|---|
| 1. | RBF (0.05 L) equipped with magnetic stirrer bar |
| 2. | Rotavapor |

### Raw Materials Consumption on 1.7 g Scale

| | | | g/batch | |
|---|---|---|---|---|
| Name | Mw | Equivalent | (ml/batch) | |
| methyl4[(3{2[2(3{[(tertbutoxy)car- | 770.89 | 1.00 | 1.7 | g |
| bonyl]amino}propoxy)ethoxy]eth- | | | | |
| oxy}propyl)carmoyl]-2-{[4-(methyl- | | | | |
| amino)phenyl]formamido}butanoate | | | | |
| LiOH*H₂O | 41.96 | 2.00 | 0.185 | g |
| MeOH | — | 20 vol. | 34 | mL |

### Procedure:

To a stirred solution of methyl4[(3{2[2 (3{[(tertbutoxy)carbonyl]amino}propoxy)ethoxy]ethoxy}propyl)carmoyl]-2-{[4-(methylamino)phenyl]formamido}butanoate 1323-028 (1.7 g, 2.21 mmol, 1 Eq.) in MeOH (34 mL, 20 vol.) at room temperature was added LiOH*H₂O (0.185 g, 4.41 mmol, 2 Eq.), and the mixture was stirred at 25° C. for 4h. After reaction was completed, the reaction mixture was concentrated in vacuo

### Purity by UPLC:

97.2% (method 6 min, 254 nm, Rₜ=2.3 min, m/z=757.75[M+H]⁺)

### Note:

The dried reaction mixture was used directly to next stage without isolation.

¹H NMR (400 MHz, Methanol-d4) δ 8.59 (s, 1H), 7.83-7.71 (m, 2H), 6.92-6.86 (m, 2H), 4.85 (s, 2H), 4.46 (dd, J=3.9, 7.5 Hz, 1H), 3.63-3.53 (m, 8H), 3.48 (dt, J=6.2, 12.0 Hz, 4H), 3.31 (s, 5H), 3.20 (td, J=1.2, 6.8 Hz, 2H), 3.12 (t, J=6.8 Hz, 2H), 2.40-1.99 (m, 4H), 1.71 (h, J=6.4 Hz, 4H), 1.44 (s, 9H).

### Step: Boc Deprotection

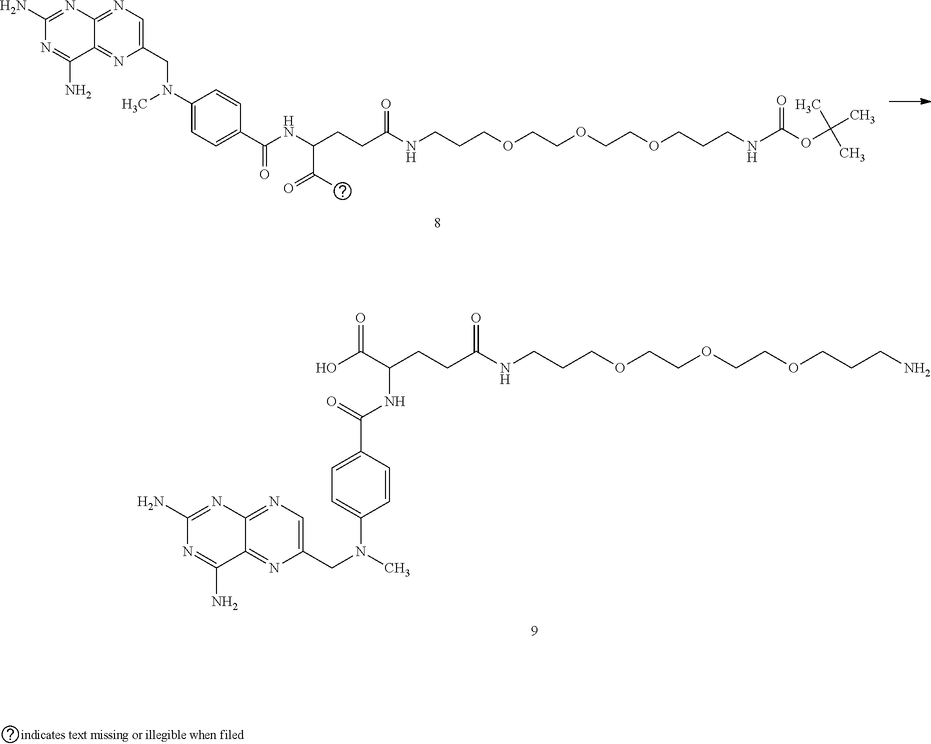 [SMILES: CC(=O)C(CCC(=O)NCCCOCCOCCOCCCNC(=O)OC(C)(C)C)NC(=O)c1ccc(N(C)Cc2cnc3nc(N)nc(N)c3n2)cc1 ; CN(Cc1cnc2nc(N)nc(N)c2n1)c1ccc(C(=O)NC(CCC(=O)NCCCOCCOCCOCCCN)C(=O)O)cc1]

### Reaction Conditions:

| Experiment performed | | | Results | | | |
|---|---|---|---|---|---|---|
| No. | Type | Conditions | Amount | Purity | Yield | Others |
| 1. | 1323-047 | The crude from 1323-033 | 0.15 g | 98.3% | 30%. | orange |
| | | (0.58 g 0.76 mmol, 1Eq), | | | | solid |
| | | in 17% HCl in water | | | | |
| | | (13 mL, 20 vol.), | | | | |
| | | 25° C., 3 h. | | | | |
| 2. | 1323-048 | The crude from | 0.36 g | 98.02% | 39.81% | orange |
| | | 1323-048-1^{st}-step (1.3 | | | | solid |
| | | mmol, 1Eq), in 17% | | | | |
| | | HCl in water (20 mL, | | | | |
| | | 20 vol.), 25° C., 3 h. | | | | |
| 2. | 1323-049 | The crude from | 1.59 g | | | |
| | | 1323-049-1^{st}-step (6.16 | | | | |
| | | mmol, 1Eq), in 17% | | | | |
| | | HCl in water (95 mL, | | | | |
| | | 20 vol.), 25° C., 3 h. | | | | |

### Apparatus

| No | Name |
|---|---|
| 1. | RBF (0.25 L) equipped with magnetic stirrer bar |
| 2. | Rotavapor |

Raw materials consumption on 4.7 g scale

| | | | g/batch | |
|---|---|---|---|---|
| Name | Mw | Equivalent | (ml/batch) | |
| Lithiumylidene 4-[(3-{2-[2-(3- | 762.8 | 1.00 | 4.7 | g |
| {[(tertbutoxy)carbonyl]ami- | | | | |
| no}propoxy)ethoxy]ethoxy}pro- | | | | |
| pyl)carbamoyl]2[(4{[(2,4diami- | | | | |
| nopteridin6yl)methyl](methyl)ami- | | | | |
| no}phenyl)formamido]butanoate | | | | |
| 17% HCl in water | — | 20 vol. | 95 | mL |

### Procedure:

Lithiumylidene 4-[(3-{2-[2-(3-{[(tert-butoxy)carbonyl]amino}propoxy)ethoxy]ethoxy}propyl)carbamoyl]-2-[(4-{[(2,4-diaminopteridin-6-yl)methyl](methyl)amino}phenyl)formamido]butanoate (crude from 1323-049-1^{st}-step, 6.16 mmol) was dissolved in 17% w HCl in water (95 mL, 20 vol), and the resulting mixture was stirred at room temperature for 3 h. The solvent was removed in vacuo (without heating), to provide a crude oil which was purified via preparative chromatography. Pure fraction was evaporated in vacuo at 25° C. to ˜10% of starting volume, the residue was dried using lyophilization.

### Purity by LCMS:

98.02% (method Gemini Rot long 7, 243 nm, Rₜ=7.407 min, m/z=655.02[M−H]⁺)

¹H NMR (400 MHz, DMSO-d6) δ 8.57 (s, 1H), 8.29 (s, 1H), 7.85 (t, J=5.6 Hz, 1H), 7.78 (d, J=6.9 Hz, 1H), 7.70-7.62 (m, 2H), 7.44 (s, 1H), 6.87-6.74 (m, 2H), 6.62 (s, 2H), 4.78 (s, 2H), 4.11 (q, J=6.6 Hz, 1H), 3.54-3.44 (m, 10H), 3.40 (t, J=6.4 Hz, 3H), 3.21 (s, 3H), 3.08 (q, J=6.3 Hz, 2H), 2.84 (t, J=7.3 Hz, 2H), 2.11 (q, J=5.9, 6.6 Hz, 2H), 2.05-1.92 (m, 1H), 1.87 (dt, J=6.4, 13.9 Hz, 1H), 1.78 (s, 2H), 1.59 (p, J=6.5 Hz, 2H).

### Batch 1323-049-Final

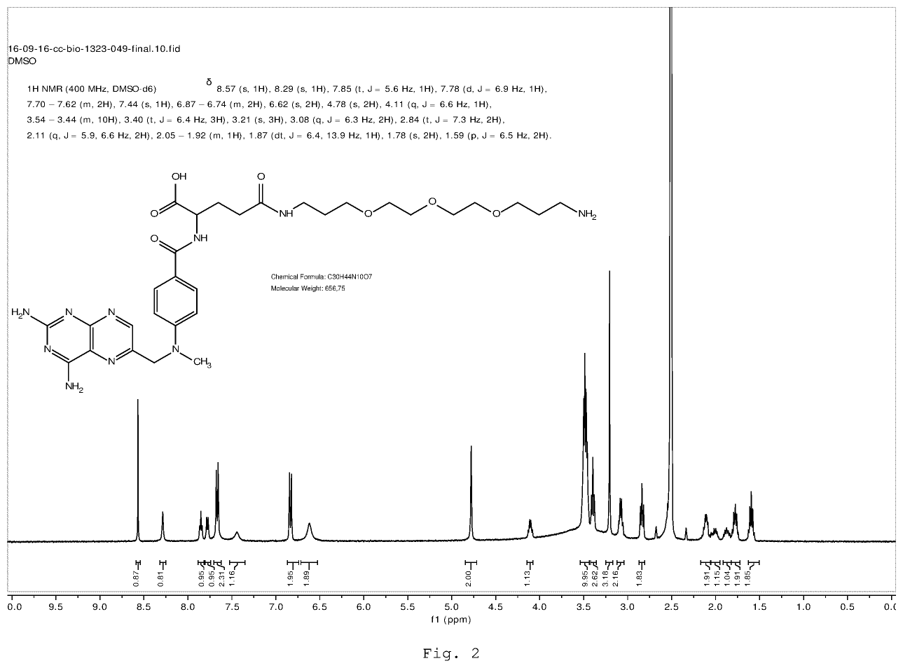

¹H NMR analysis was performed in DMSO-d6 as the solvent. ¹H NMR confirmed the structure of the product (see FIG. 2).

### Example 3—Synthesis of Modified Methotrexate-Coupled Gold Nanoparticles (MTX-GNPs)

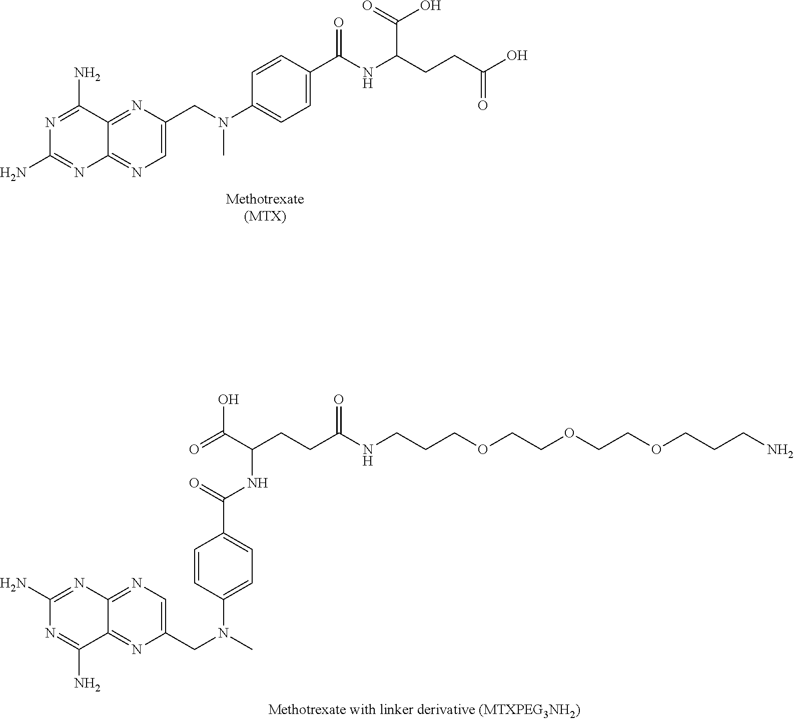 [SMILES: CN(Cc1cnc2nc(N)nc(N)c2n1)c1ccc(C(=O)NC(CCC(=O)O)C(=O)O)cc1 ; CN(Cc1cnc2nc(N)nc(N)c2n1)c1ccc(C(=O)NC(CCC(=O)NCCCOCCOCCOCCCN)C(=O)O)cc1]

The chemical name of the methotrexate derivative with linker synthesised as described in Example 2 is 4-[(3-{2-[2-(3-aminopropoxy)ethoxy]ethoxy}propyl)carbamoyl]-2-[(4-{[(2,4-diaminopteridin-6-yl)methyl](methyl)amino}phenyl)formamido]butanoic acid.

The aim of this experiment was to synthesise 50 mg GNP with MTXPEG3NH₂ (also known as MTX-(EG) 3-NH₂) loading of >12 equivalents per GNP.

The base GNP particle was ([α-GalC2]_{52%}[HSPEG₈COOH]_{48%}@Au), and the coupling was performed by using the EDC/NHS method. Note that in contrast to the positively charged AL of Example 1, the base GNP in this example has PEG₈ (i.e. (EG)₈-containing) ligands with a carboxylic acid terminal functionality (negatively charged) in addition to the α-Gal-C2 ligands. The base GNPs [α-GalC2]_{52%}[HSPEG₈COOH]_{48%}@Au were synthesised essentially as described in WO2017/017063 (see Example 5 thereof), incorporated herein by reference.

Reagents

| Material | Supplier | Batch/R number | Comments |
|---|---|---|---|
| Starting GNP | Midatech Pharma | M324-020-01 | [α-GalC2]_{52%}[HSPEG8COOH]_{48%}@Au |
| EDC | Sigma-Aldrich | SLBT0569 | — |
| NHS | ALDRICH | MKBX1364V | — |
| HEPES | Sigma | SLBM8525V | pH = 7.83, 40 mM |
| MTXPEG3NH₂ | SELVITA | BIO-1323-049-final | started with 60 eq per NP |

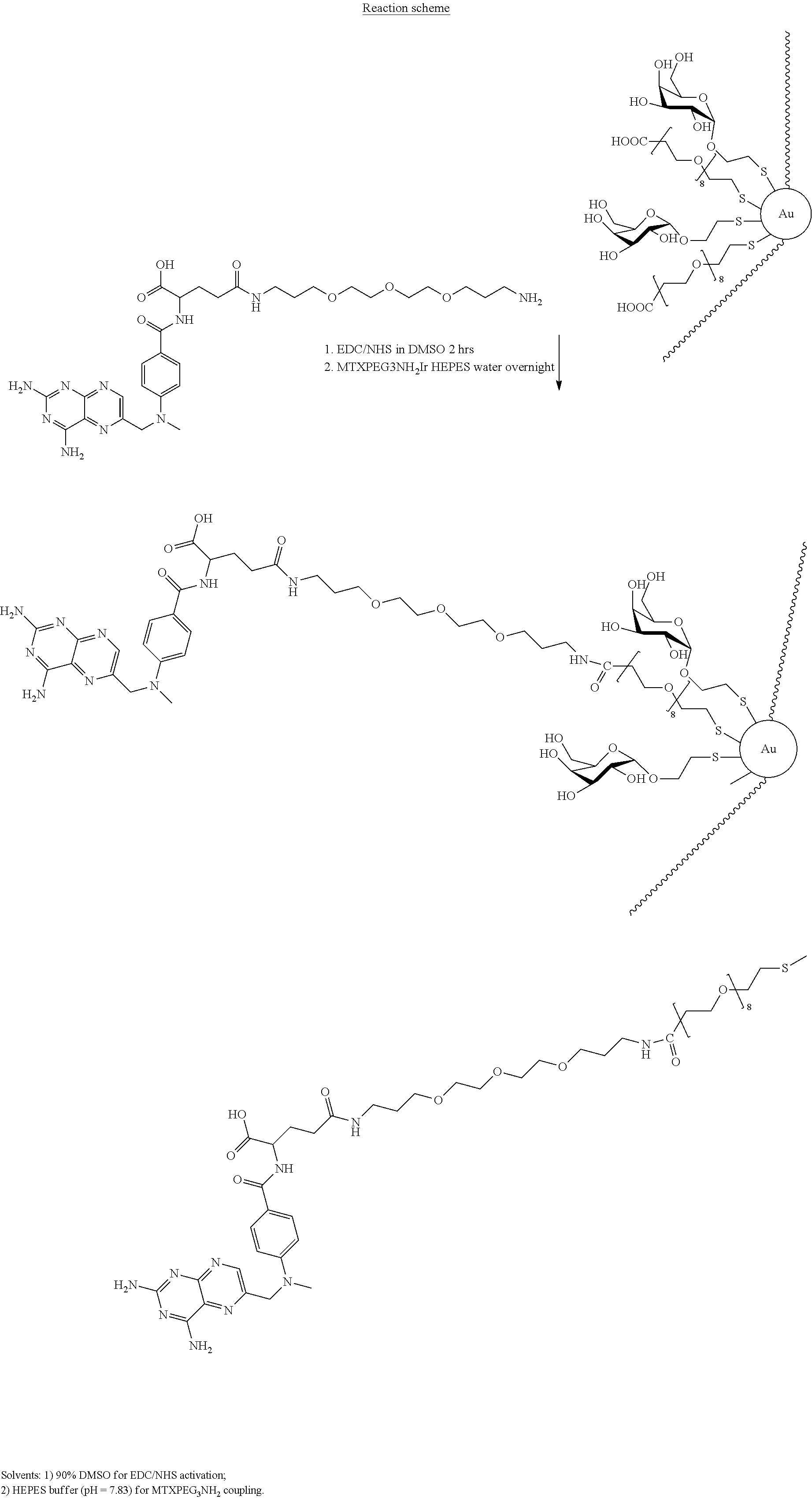

EDC/NHS activation

38.12 mg of EDC was dissolved in 3.31 mL DMSO first, then 3.16 mL of this 60 mM EDC DMSO stock was mixed with 43.67 mg of NHS to give a final DMSO stock of EDC (60 mM)/NHS (120 mM).

11 mL 90% DMSO GNP solution (60 mg Au) was kept stirring at 500 rpm, then 2.79 mL of EDC/NHS DMSO stock was added dropwise. The reaction mixture was kept stirring at 500 rpm at R.T for 2 hr ([Au]≈4.35 mg/mL).

After two hours activation, the GNP-NHS DMSO solution was concentrated in 8×15 mL Amicon tubes (10K) by centrifugation (4300 rpm, 8 min). The GNP final concentration was about 12 mL.

MTXPEG₃NH₂ Coupling:

$${MTXPEG}_{3} {NH}_{2} ( 60 eq per NP ) : \frac{60 mg Au}{196.97} \div 100 \times 60 \times 656.75 = 120 mg$$

120 mg of MTXPEG3NH₂ was first dissolved in 20 mL HEPES buffer (pH=7.83), this was then transferred to a 250 mL round bottomed flask. While stirring at 600 rpm at RT (˜22° C.), the 12 mL concentrated GNP-NHS solution was added dropwise. Then, 20 mL of HEPES buffer was added into this mixture. The reaction mixture was stirred at 600 rpm at RT (˜22° C.) overnight ([Au]=1.15 g/L).

The next morning, the reaction solution mixture was concentrated in 15 mL Amicon tubes (10K), and purified by washing with Milli-Q water (×8, 4300 rpm, 8 min per wash). The concentrated solution was then spun at 13.3G for 5 min (×2) to remove any large size particles from solution. The final concentrated GNP solution was diluted with Milli-Q water to give a final volume of 11 mL.

### Chemical and Physical Analysis

| | | Zeta | |
|---|---|---|---|
| [Au] | Size | Potential | |
| (μg/μl) | (nm) | (mV) | UV-VIS |
| 3.889 | 5.678 | −22.8 | No plasmon band |
| | | | at 520 nm |

MTXPEG₃NH₂ content was assessed by Agilent HPLC with the following sample preparation: 8 μg Au was diluted with 0.2M TCEP to give a final volume of 40 μL ([Au]=0.2 g/L), then incubated at 37° C. and agitated at 600 rpm for 1 hr. After incubation, 40 μL of Milli-Q water was added to give a final total volume of 80 μL ([Au]=0.1 g/L). This solution was analysed by HPLC, (20 μL injection→2 μg Au). For MTXPEG₃NH₂ standards: 4 μL of 2 g/L MTXPEG₃NH₂ aqueous stock solution and 36 μL of 0.2M TCEP were incubated at 37° C. and agitated at 600 rpm for 1 hr. To this, 160 μL Milli-Q water was added (total volume=200 μL [MTXPEG₃NH₂]=0.04 g/L). This solution was analysed by HPLC, (10 μL injection→0.4 μg, 20 μL→0.8 μg and 30 μL→1.2 μg).

A standard curve was generated (taking into account the effect of the yellow MTXPEG₃NH₂ compound upon colorimetric gold quantification and thereby correcting the gold concentration). MTXPEG₃NH₂ loading was determined to be 16.7 equivalents per GNP, with incorporation of 97.4%.

In summary, this batch of MTXPEG₃NH₂ particles had the following properties: small size (5.678 nm) with a single size population, negative Zeta potential (−22.8 mV), no plasmon band at 520 nm, MTXPEG₃NH₂ incorporation on GNP was 97.4%, and the loading on the final particles was 16.7 eq per GNP (mean). Consistent results were also found between batches at different reactor sizes (50 mg and 100 mg Au). These results compare favourably to the results obtained in Example 1. In particular, the modified MTX (MTXPEG₃NH₂) facilitated significantly higher loading (16.7 equivalents vs. around 5 equivalents for MTX), high loading efficiency (97.4%) and a single size population. Without being bound by any particular theory, the present inventors consider that the MTXPEG₃NH₂ coupling to the PEG₈COOH ligands of the GNPs avoids the issue of multiple carboxyl sites on MTX described in Example 1 and that this may explain the observed difference between single size distribution/population (Example 2) and two size distributions/populations (Example 1). Moreover, the loading efficiency of 97.4% determined here is markedly higher than even the highest loading efficiency of 83±2% reported in Bessar et al., 2016. The loading of Bessar et al., 2016 in terms of equivalents of MTX per GNP is not reported. However, the weight ratio of Au-3MPS to MTX drug used in the synthesis of Bessar et al., 2016 was 5:1 (i.e. excess of GNPs).

In conclusion, the [α-GalC2][MTXPEG₃NH—CO-PEG₈]@Au GNPs exhibit high MTX loading in comparison to that observed for the unmodified MTX of Comparative Example 1.

### Example 4—Formulation of [α-GalC2][MTXPEG₃NH—CO-PEG₈]@Au GNPs into Hydrogels

Currently available marketed topical formulations of methotrexate exhibit poor penetration through the stratum corneum due to the hydrosoluble nature of the drug, which is mostly in a dissociated form at physiological pH (pH 6). The ultra-small size (<5 nm) of the GNPs disclosed herein having a corona comprising carbohydrate ligands, which allows for suitable net surface charge, may offer potential for increasing the capacity of methotrexate penetration across intact skin.

Recently, a topical gold nanoparticle cream formulation was reported by Bessar et al. 2016 to show preliminary proof of percutaneous adsorption of methotrexate conjugated GNP. Hydrogels have also been applied for the development of topical nanoparticle formulations, as these provide a single-phase vehicle that could allow greater flexibility and control of drug delivery from the formulation. In addition, hydrogels offer the advantage of rapid evaporation leaving no residual formulation on the skin compared to commercially available ointments, in which high affinity between drug and formulation base compromises efficient drug transfer into the skin. Therefore, Carbopol hydrogels were selected for the development of GNP based topical formulations.

The following polymers (Lubrizol Corporation) were evaluated: Carbopol® ETD 2020 (C10-30 alkyl acrylate cross polymer), Carbopol® 980 NF polymer and Carbopol® 974P NF Polymer. Gels were prepared by dispersing 1-3% w/v of Carbopol polymer (w/v) into purified water with constant mixing and thus allowed to hydrate for 5 hours. Care was taken to avoid air entrapment by agitating the solution slowly on a rocker during preparation of the gel. After 5 hours, the pH of the gel was adjusted to pH 7.4 using triethanolamine (Sigma-Aldrich, Lot #STBF616V) to neutralise the pH and turn the solution into a gel. A 2% Carbopol® 980 gel was found to produce a clear, homogenous gel whereas ETD 2020 gel was more difficult to produce homogeneity. Therefore, formulation of the gold glyconanoparticles into a hydrogel proceeded with the Carbopol® 980 NF polymer.

MTXPEG₃NH₂-loaded GNPs were prepared essentially as described in Example 2. For production of methotrexate-GNP hydrogel, 2% w/v Carbopol®980 was initially dispersed for 5 hours with constant mixing. The MTX-PEG₃-NH₂-loaded GNPs were concentrated using Amicon centrifugal filter tubes (10 K membrane molecular weight cut-off) with centrifugation at 5000 rpm for 10 min. Prior to addition to the 2% Carbopol®980 solution, the pH of MTX-PEG₃-NH₂-loaded GNPs was adjusted to pH 2.6. The acidic MTX-PEG₃-NH₂-loaded GNPs were then added to the 2% Carbopol®980 solution. However, the nanoparticles were observed to precipitate rapidly in the Carbopol®980 solution. Plain methotrexate drug gel was prepared by dissolving MTX-PEG₃-NH₂ in water and adjusting the pH to pH 4.5. The MTX-PEG₃-NH₂ solution was added to the previously made 2% Carbopol®980 solution. However, a small level of yellow precipitation was also observed.

The method for formulating gold nanoparticles into Carbopol®980 gels was optimised by testing the effects of pH and speed of addition of nanoparticles using control [α-Gal][PEG₈COOH]@Au GNPs. Homogenous nanoparticle gels without precipitation were obtained when the pH of the Carbopol®980 solution was adjusted to pH 7.4 prior to the drop-wise addition of the [α-Gal][PEG₈COOH]@Au GNPs with constant mixing. Similarly, for methotrexate gel (without nanoparticles), a homogenous yellow gel without precipitation was obtained when the pH of the Carbopol®980 solution was adjusted to pH 7.4 prior to the drop-wise addition of modified methotrexate. The gels were all stored at 4° C.

For production of methotrexate-GNP hydrogel, 2% w/v Carbopol®980 was dispersed for 5 hours with constant mixing. The pH of the Carbopol®980 solution was adjusted to pH 7.4 to produce a clear gel. MTX-PEG₃-NH₂-loaded GNPs were concentrated using Amicon centrifugal filter tubes and then added to the 2% Carbopol®980 gel. The resulting MTX-PEG₃-NH₂-loaded GNP hydrogel was a homogeneous brown gel, with no precipitation of MTX-PEG₃-NH₂-loaded GNPs observed in the gel. Control GNP (no drug) gel was also prepared using [α-Gal-C2][PEG₈COOH]@Au GNPs and found to produce a brown, homogenous gel. Plain methotrexate drug gel was prepared by adding MTX-PEG₃-NH₂ dissolved in water to the pH 7.4 adjusted Carbopol®980 gel (2%). The methotrexate was found to be incorporated readily, producing a yellow homogenous hydrogel, with no precipitation of the methotrexate derivative observed.

The concentration of MTX-PEG₃-NH₂ in the MTX-PEG₃-NH₂-loaded GNP hydrogel was in the range 0.18-0.2% (w/w).

MTX concentration in previously reported topical formulations are generally in the range 0.25% to 0.5% (see, e.g., Lakshmi et al., Indian J Dermatol Venereol Leprol, 2007, Vol. 73, pp. 157-161 and Jabur et al., J Fac Med Baghdad, 2010, Vol. 52, No. 1, pp. 32-36).

### Example 5—Cytostatic Activity of MTX-PEG₃-NH₂-Loaded GNPs on HEP3B and U87MG Cells

The cytotoxic or cytostatic activity of methotrexate, the linker-modified methotrexate, MTX-(EG)₃-NH₂ and of MTX-(EG)₃-NH₂-loaded gold nanoparticles (GNPs) ([MTX-(EG)₃-NHC(O)-(EG)₈][alpha-Gall][COOH-(EG)₈]@Au) was assessed by treatment of Hep3B and U87MG cells. Incubation time was 72 hours and the concentration range was 40 pM to 500 μM.

The results are expressed as IC₅₀ and are as follows:

| Test agent | Hep3B cells | U87MG cells |
|---|---|---|
| MTX | ++ | ++ |
| MTX-(EG)₃-NH₂ | ++ | + |
| MTX-(EG)₃-NH₂-loaded GNPs | + | + |

++ 1 nm < x < 1 μM
+ 1 μM < x < 1 mM

These results demonstrate that the linker-modified MTX alone or when coupled to a GNP exhibits cytotoxic or cytostatic activity against the test cell lines.

### Example 6—Synthesis of N-[4-[[(2,4-diamino-6-pteridinyl)methyl]methylamino]benzoyl]-L-glutamoyl-[1-amino-4,9-dioxa-12-dodecane]

### 2,4-diamino-6-(hydroxymethyl)pteridine

2,4-diamino-6-(hydroxymethyl)pteridine hydrochloride (3.34 g, 14.6 mmol, 1.00 eq) was suspended in H₂O (120 mL) and resulting mixture was heated to 40° C. After 30 min of stirring at 40° C. suspension was still observed. The mixture was cooled to room temperature and 1 M NaOH (12 mL) was dropped at room temperature within 5 min. A yellow solid precipitated. The obtained mixture was stirred for 30 min at room temperature. The yellow solid was filtered, washed with water (2×60 mL) and dried under reduced pressure at 40° C. to afford the 2,4-diamino-6-(hydroxymethyl)pteridine (2.4 g, yellow solid) which used in the next step without further purification.

### N-[4-[[(2,4-diamino-6-pteridinyl)methyl]methylamino]benzoic Acid

2,4-diamino-6-(hydroxymethyl)pteridine (2.4 g) was suspended in anhydrous DMA (20 mL) and triphenylphosphine dibromide (13.87 g, 32.9 mmol, 2.25 eq) was added. The suspension was stirred at room temperature for 20 h before adding 4-(methylamino)benzoic acid (2.45 g, 16.2 mmol) and DIPEA (5.34 mL, 30.7 mmol). The resulting suspension was stirred at room temperature for 48 h until no bromide was evident by UPLC analysis. The dark reaction mixture was poured into 0.33 M NaOH (190 mL), the precipitate removed by filtration and the filtrate acidified to approximately pH 4.5 with 10% AcOH aqueous solution (20 mL). The precipitate was collected by filtration, washed with water (2×100 mL) and triturated with methanol (23 mL) at 40° C. to give a dark yellow solid that was dried under reduced pressure at 40° C. to afford the desired product (3.73 g, 78% yield, UPLC: 92%)

### α-t-Butyl-N-[4-[[(2,4-diamino-6-pteridinyl)methyl]methylamino]benzoyl]-L-glutamate

A suspension of pteroic acid (2.0 g, 6.1 mmol, 1.0 eq) in DMF (anhydrous, 40 mL) and triethylamine (1.7 mL, 12.3 mmol, 2.0 eq) was treated with TPTU (1.83 g, 6.1 mmol, 1.0 eq) and the suspension was stirred at room temperature for 2.5 h. In a separate flask a suspension of L-glutamic acid-α-t-butyl ester (1.44 g, 7.1 mmol, 1.15 eq) and triethylamine (1.0 mL, 7.4 mmol, 1.2 eq) in DMF (anhydrous, 30 mL) was prepared. The active ester was slowly added to the suspension of the glutamic acid and the resulting mixture was stirred at room temperature for 72 h. DMF was removed under reduced pressure at 50° C. The residue, a dark brown oil, was treated with ethyl acetate (70 mL) and resulting mixture was stirred for 30 min, the resulting yellow solid was collected by filtration and triturated with chloroform (70 mL). The solid was collected by filtration, washed with chloroform (2×30 mL) and dried under reduced pressure (2 h) to afford the desired product (2.78 g, yield 89%)

### α-t-Butyl-N-[4-[[(2,4-diamino-6-pteridinyl)methyl]methylamino]benzoyl]-L-glutamoyl-[1-(t-butyloxycarbonyl-amino)-4,9-dioxa-12-dodecane]

α-t-Butyl-N-[4-[[(2,4-diamino-6-pteridinyl)methyl]methylamino]benzoyl]-L-glutamate (5.76 g, 11.3 mmol, 1.0 eq) was dissolved in DMF (anhydrous, 115 mL) and triethylamine (5.0 mL, 36.1 mmol, 3.2 eq) was added. TPTU (3.35 g, 11.3 mmol, 1.0 eq) was added and the solution was stirred at room temperature for 30 min. A solution of 1-(t-Butyloxycarbonyl-amino)-4,9-dioxa-12-dodecanamine (4.34 g, 13.5 mmol, 1.2 eq) in DMF (anhydrous, 86.0 mL) was added. The resulting mixture was stirred at room temperature for 72 h and the DMF was removed under reduced pressure at 50° C. to afford the crude material (14.6 g) as a brown oil.

### N-[4-[[(2,4-diamino-6-pteridinyl)methyl]methylamino]benzoyl]-L-glutamoyl-[1-amino-4,9-dioxa-12-dodecane]

The protected amine (0.07 g, 0.1 mmol, 1.0 eq) was dissolved in TFA (4 mL) and stirred at room temperature for 16 h. The solvents were removed (without heating) to give an orange-red oil that was treated with diethyl ether (10 mL) and after few min a yellow solid was formed. Suspension was stirred for 1h at room temperature and filtered to give an extremely hygroscopic solid that was dissolved in mixture of ACN, water and DMSO and purified by preparative HPLC Step_3_product (2.70 g, 3.3 mmol, 1.0 eq) was dissolved in TFA (5.1 mL, 20 eq). Reaction was carried out at room temperature overnight, diluted with DCM and evaporated to dryness (without heating) to afford the crude product as a TFA salt as a brown oil (6.4 g) that was purified by preparative HPLC (Shimadzu LC-20AP with a Gemini-NX 5 μm C18 (250×21.2 mm), 110 Å column)
-oOo-

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only and are not to be construed as limiting the disclosure in any way.

1. A nanoparticle comprising:
a core comprising a metal and/or a semiconductor; and
a plurality of ligands covalently linked to the core, wherein said ligands comprise:
(i) at least one dilution ligand comprising a carbohydrate, glutathione or an ethylene glycol-containing moiety; and
(ii) a ligand of the formula D-L₁-Z-L₂, wherein D comprises an antifolate drug or folic acid, L₁ comprises a first linker portion comprising a C2-C12 glycol and/or C1-C12 alkyl chain, L₂ comprises a second linker portion comprising a C2-C12 glycol and/or C1-C12 alkyl chain, wherein L₁ and L₂ may be the same or different, and wherein Z represents a divalent linker group of up to 10 atoms linking L₁ and L₂, and wherein Z comprises at least 2 heteroatoms and L₂ is coupled to said core.

2. The nanoparticle of claim 1, wherein D comprises the following structure; 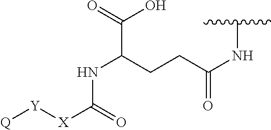
wherein;
X is a 3 to 8 membered carbocyclic, heterocyclic, carboaromatic or heteroaromatic ring,
Y is a linker group having 1 to 20 atoms comprising one or more atoms selected from H, C, N, O and S;
wherein Y is optionally substituted by one or more groups having 1 to 20 atoms comprising of one or more atoms selected from H, C, N, O and S, and
Q is a fused bicyclic heterocyclic or heteroaromatic ring optionally substituted with one or more groups selected from amino, hydroxyl, carbonyl, methyl, ethyl, propyl, isopropyl, butyl and isobutyl.

3. The nanoparticle of claim 2, wherein D comprises an antifolate drug selected from the group consisting of: methotrexate, pemetrexed, ralitrexed and pralatrexate.

4. The nanoparticle of claim 3, wherein D is selected from the following structures;

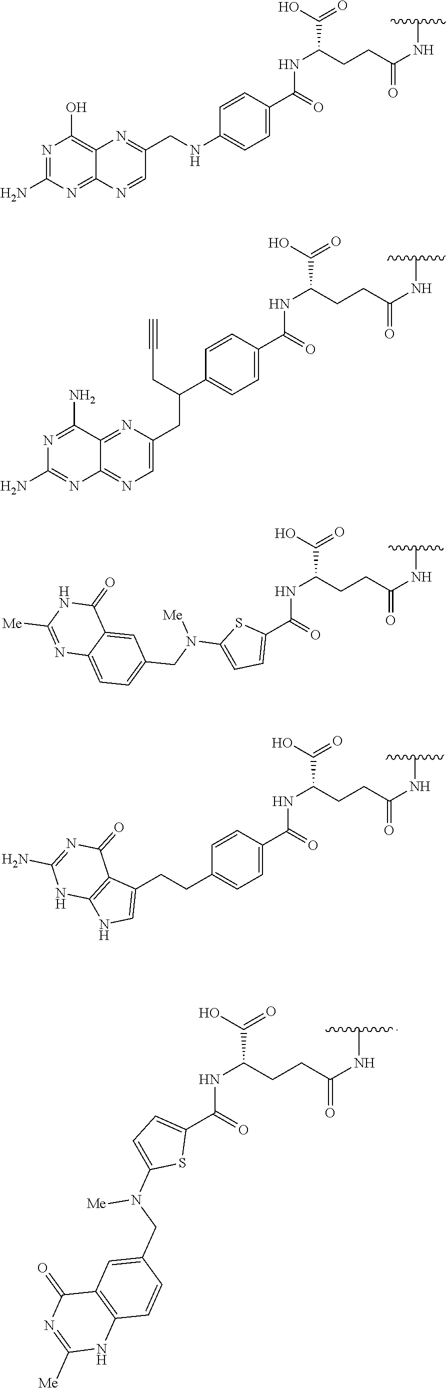 [SMILES: *NC(=O)CC[C@H](NC(=O)c1ccc(NCc2cnc3nc(N)nc(O)c3n2)cc1)C(=O)O ; *NC(=O)CC[C@H](NC(=O)c1ccc(C(CC#C)Cc2cnc3nc(N)nc(N)c3n2)cc1)C(=O)O ; *NC(=O)CC[C@H](NC(=O)c1ccc(N(C)Cc2ccc3nc(C)[nH]c(=O)c3c2)s1)C(=O)O ; *NC(=O)CC[C@H](NC(=O)c1ccc(CCc2c[nH]c3[nH]c(N)nc(=O)c23)cc1)C(=O)O ; *NC(=O)CC[C@H](NC(=O)c1ccc(N(C)Cc2ccc3[nH]c(C)nc(=O)c3c2)s1)C(=O)O]

5. The nanoparticle of claim 1, wherein Z comprises a 3-10 membered carboaromatic, a 3-10 membered carbocycle, a 3-10 membered heterocycle, a 3-10 membered heteroaromatic, an imide, an amidine, a guanidine, a 1,2,3-triazole, a sulfoxide, a sulfone, a thioester, a thioamide, a thiourea, an amide, an ester, a carbamate, a carbonate ester or a urea.

6.-7. (canceled)

8. The nanoparticle of claim 1, wherein D-L₁-Z-L₂ is of the formula:

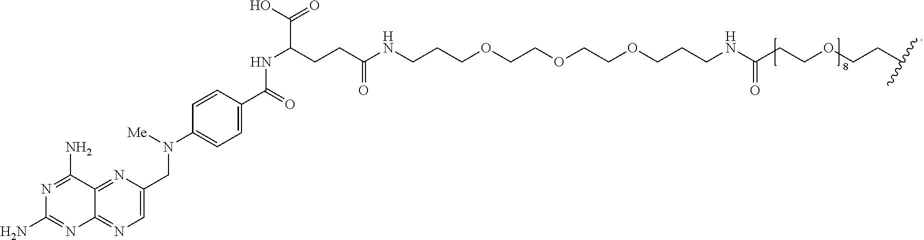

9. The nanoparticle of claim 1, wherein D-L₁-Z-L₂ is of the formula:

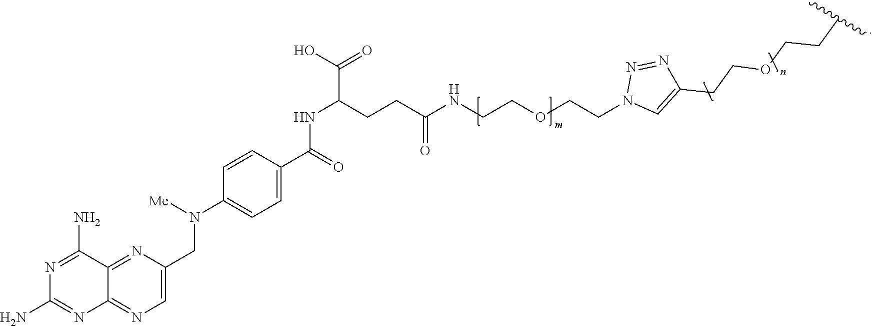

10. The nanoparticle of claim 1, wherein D-L₁-Z-L₂ is of the formula:

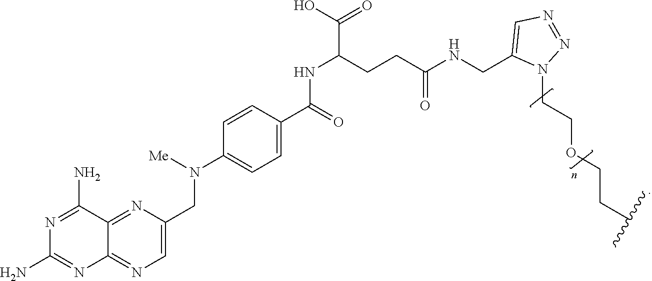

11. The nanoparticle of claim 1, wherein D-L₁-Z-L₂ is of the formula:

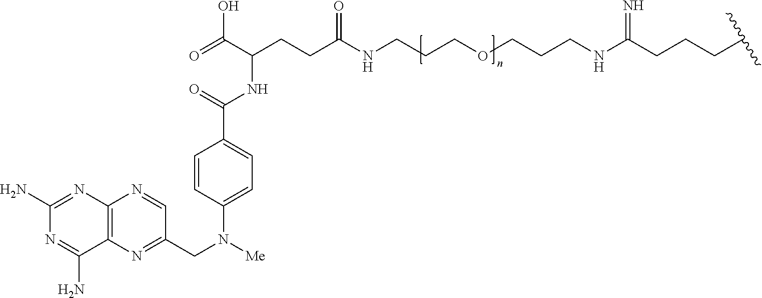

12. The nanoparticle of claim 1, wherein L₂ is bound to the core via a terminal sulphur atom.

13. The nanoparticle of claim 12, wherein D-L₁-Z-L₂ is of the formula:

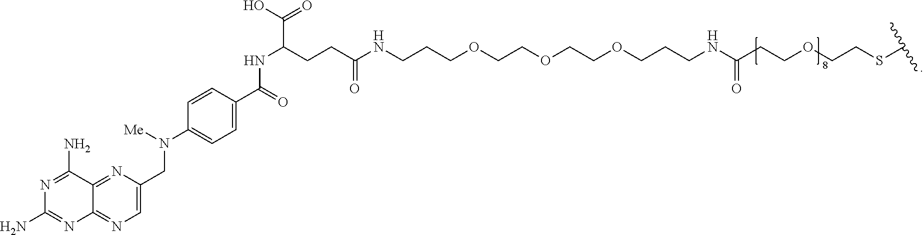

14. The nanoparticle of claim 12, wherein D-L₁-Z-L₂ is of the formula: 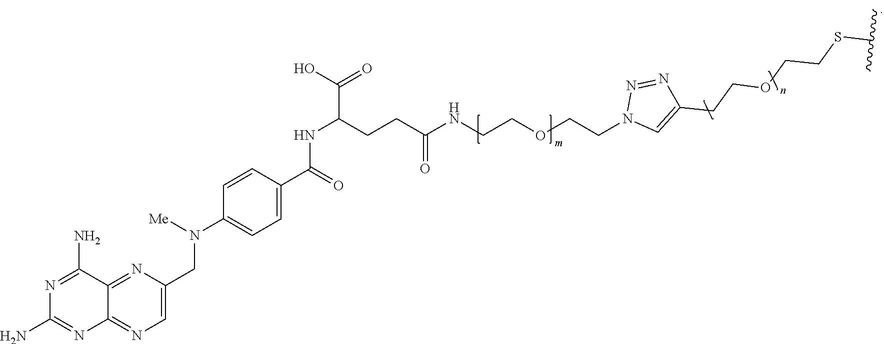
wherein n and m are independently 1, 2, 3, 4, 5, 6, 7, 8, 9 or 10.

15. The nanoparticle of claim 12, wherein D-L₁-Z-L₂ is of the formula: 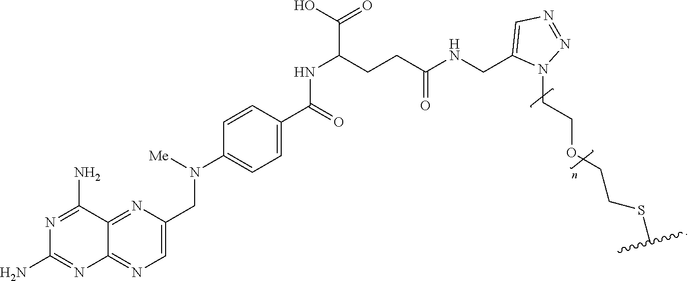
wherein n is an integer of between 1 and 15.

16. The nanoparticle of claim 12, wherein D-L₁-Z-L₂ is of the formula: 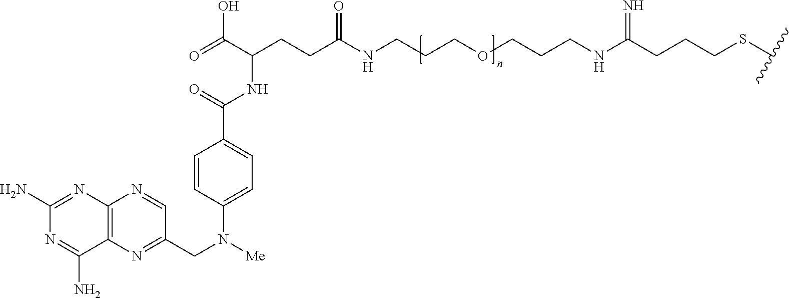
wherein n is an integer of between 1 and 15.

17.-29. (canceled)

30. The nanoparticle of claim 1, wherein said plurality of ligands further comprises a therapeutically active agent or a detectable moiety.

31. The nanoparticle of claim 30, wherein the therapeutically active agent comprises an anti-cancer agent.

32. The nanoparticle of claim 31, wherein the anti-cancer agent is selected from the group consisting of: a maytansinoid, doxorubicin, irinotecan, Platinum (II), Platinum (IV), temozolomide, carmustine, camptothecin, docetaxel, sorafenib, monomethyl auristatin E (MMAE) and panobinostat.

33. A pharmaceutical composition comprising a plurality of nanoparticles of claim 1 and at least one pharmaceutically acceptable carrier or diluent.

34. The pharmaceutical composition of claim 33, wherein the pharmaceutical composition is in the form of a gel.

35.-41. (canceled)

42. A method of treating a proliferative disorder, an inflammatory disorder or an autoimmune disease in a mammalian subject, comprising administering a nanoparticle according to claim 1 to the subject in need of therapy.

43.-48. (canceled)

49. A process for the production of a compound of the formula D-L₁-R₁, wherein D comprises an antifolate drug or folic acid, L₁ comprises —(OCH₂CH₂)ₚ—, wherein p is an integer in the range 1 to 10, and wherein R₁ comprises an amine group, the process comprising the following steps; 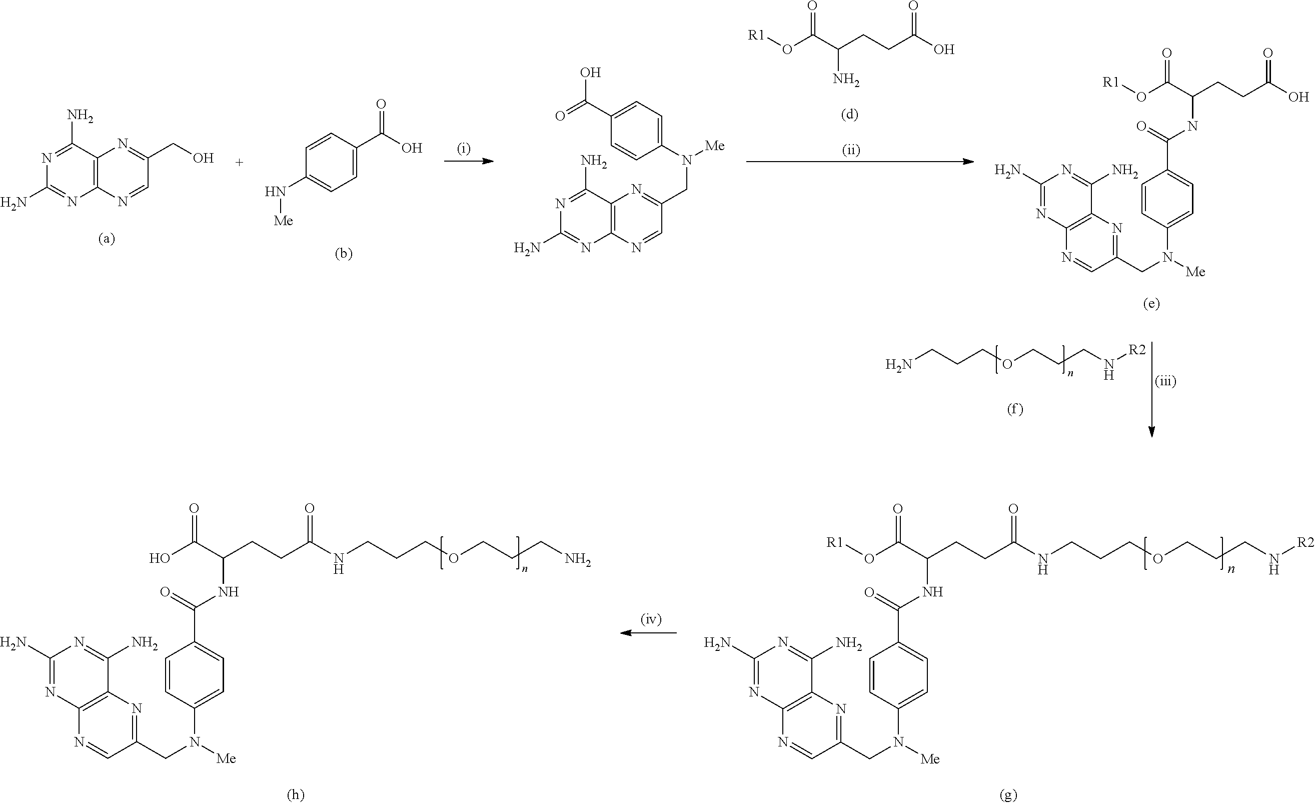
(i) halogenation of an alcohol of formula (a) to afford a halogen compound (a1) that is used in a displacement reaction with an amine of formula (b) to afford an compound of formula (c);
(ii) performing an amide coupling with compounds of formulae (c) and (d) to afford an amide of formula (e),
(iii) performing an amide coupling with compounds of formulae (e) and (f) to afford an amide of formula (g),
(iv) removing the amine and carboxylic acid protecting groups of the compound of formula (g) to afford a compound of formula (h),
wherein R1 is a carboxylic acid protecting group and R2 is an amine protecting group.

50.-57. (canceled)

58. A method of treating a proliferative disorder, an inflammatory disorder or an autoimmune disease in a mammalian subject, comprising administering a pharmaceutical composition according to claim 33 to the subject in need of therapy.
